(19) United States
(12) Patent Application Publication
(10) Pub. No.: US 2006/0292213 A1
(43) Pub. Date: Dec. 28, 2006

(54) Enoximone formulations and their use in the treatment of PDE-III mediated diseases

(75) Inventors: Michael J. Gerber, Denver, CO (US); Rick Gorczynski, Westminster, CO (US); Michael Bristow, Englewood, CO (US)

Correspondence Address: FULBRIGHT & JAWORSKI L.L.P., 600 CONGRESS AVE., SUITE 2400, AUSTIN, TX, 78701, US

(73) Assignee: Myogen, Inc.

(21) Appl. No.: 11/138,894

(22) Filed: May 26, 2005

Related U.S. Application Data
(60) Provisional application No. 60/582,194, filed on Jun. 23, 2004.

(51) Int. Cl. A61K 31/4166; A61K 9/48

(52) U.S. Cl. 424/451; 514/389

(57) ABSTRACT

The present invention provides pharmaceutical formulations of the drug enoximone for use in treatment of disease states in which inhibition of PDE-III may be beneficial.

This application claims benefit of priority to U.S. Provisional Application Ser. No. 60/582,194 filed Jun. 23, 2004, the entire contents of which are hereby incorporated by reference.

### BACKGROUND OF THE INVENTION

1. Field of the Invention

The present invention relates generally to novel formulations of the drug enoximone for use in treating a variety of disease states. More particularly, the invention relates to the treatment of diseases where inhibition of the enzyme phosphodiesterase-III (PDE-III) would be beneficial.

2. Description of Related Art

Phosphodiesterases (PDEs) are a class of intracellular enzymes involved in the metabolism of the second messenger nucleotides, cyclic adenosine monophosphate (cAMP), and cyclic guanosine monophosphate (cGMP) (see, Doherty, 1997). Numerous phosphodiesterase inhibitors have previously been described in the literature for a variety of therapeutic uses, including treatment of obstructive lung disease, allergies, hypertension, angina, congestive heart failure and depression (see, Goodman and Gilman's, Chapter 34). Oral and parenteral administration of PDE-V inhibitors, as alluded to above, have also been used for the treatment of erectile dysfunction (Doherty, supra; see also PCT Publication Nos. WO 96/16644 and WO 94/28902).

As explained by Komas et al. (1996), those initially working in the field partially purified what was believed to be a single enzyme responsible for specifically hydrolyzing the 3′-bond of cyclic nucleotides. However, it later became clear that multiple forms of phosphodiesterase inhibitors were present in different tissues; the enzymes were classified into three major groups, one of which exhibited high affinity for cAMP and designated as the “low Kₘ” cAMP PDE. This “low Kₘ” cAMP PDE was ultimately discovered to consist of two distinct isoenzymes having entirely different properties, including physical properties, kinetic characteristics and inhibitor specificities. One isoenzyme was found to be very sensitive to inhibition by cilostamide and cGMP, and is now known as the cAMP-specific, cGMP-inhibited cyclic nucleotide phosphodiesterase (cGI-PDE) or PDE III, while the second isoenzyme was classified as PDE IV (Komas et al., 1996).

The phosphodiesterases have now been classified into ten major families, Types I-X, based on amino acid or DNA sequences. The members of the family vary in their tissue, cellular and subcellular distribution, as well as their links to cAMP and cGMP pathways. For example, the corpora cavernosa contains: Type III phosphodiesterases, which as explained above are cAMP-specific cGMP inhibitable; Type IV phosphodiesterases, the high affinity, high-specificity cAMP-specific form; and Type V phosphodiesterases, one of the cGMP-specific forms.

Various compounds in addition to enixomone are known as inhibitors of phosphodiesterases, including vinpocetine, milrinone, amrinone, pimobendan, cilostamide, piroximone, vesnarinone, rolipram, RO20-1724, zaprinast, dipyridamole, pentoxifylline, sildenafil citrate (Viagra®), doxazosin, papaverine, prazosin, terazosin, trimazosin and hydralazine. PCT Publication No. WO 94/28902 discloses a series of pyrazole [4,3-d] pyrimidin-7-ones cGMP phosphodiesterase inhibitors. PCT Publication No. WO 96/16644 also discloses a variety of cGMP phosphodiesterase inhibitors, including griseolic acid derivatives, 2-phenylpurinone derivatives, phenylpyridone derivatives, fused and condensed pyrimidines, a pyrimdopyrimidine derivative, a purine compound, a quinazoline compound, a phenylpyrimidone derivative, an imidazoquinoxalinone derivative or aza analogues thereof, a phenylpyridone derivative, and others.

PDE-III has been implicated as a target molecule for therapy in a variety of diseases. Cardiac hypertrophy, for example, is one such disease for which inhibition of PDE-III is indicated. Cardiac hypertrophy has been established as an independent risk factor for cardiac morbidity and mortality (Levy et al., 1990). Type III PDE's, along with type V, if inhibited, are also known to affect the human corpus cavernosum (Stief et al., 1998). For example, the hydrolysis of the second messenger cyclic AMP by PDE-III is known to play an important regulatory role in the relaxation of cavernous smooth muscle of the penis (Kuthe et al., 1999). Thus, inhibition of PDE-III is suggested for treatment of erectile dysfunction (ED).

Recently, Scottish researchers have investigated the mechanism by which PDE-III activity is increased following chronic hypoxia. PDE-IIIA was found to be over-expressed through a protein kinase A-dependent mechanism. The data implicates PDE-III in the pathophysiology of pulmonary hypertension, delineating new strategies for targeting this enzyme and supporting the use of such strategies as therapeutic approaches (Murray et al., 2002).

PDE-III is also known to affect platelet aggregation and PDE-III inhibitors may be of use in treating platelet disorders, coagulation and agglutination disorders (Sly et al., 1997). It has been reported that inhibition of PDE-III may be beneficial to alleviate the symptoms of angina (Schlepper et al., 1991). There are a number of reports indicating that PDE-III inhibition could be beneficial in the treatment of renal diseases (Wang et al., 2002; Wagner et al., 1998; Tsuboi et al., 1996; and Takeda et al., 1991). Yamaura et al. (2001) have shown that PDE-III inhibition may be useful in the treatment of gastrointestinal disorders. Finally, inhibition of PDE-III has also been indicated for a variety of vascular and circulatory disorders (Ichioka et al., 1998; Shiraishi et al., 1998; and Boldt et al., 1993).

Therefore, improving PDE-III inhibition therapy is highly desirable given the widespread involvement of PDE-III in disease states. Given the failure of PDE-III inhibitor clinical trials in the 1980's and 1990's due to alleged lethality or lack of efficacy in a variety of indications, exploration for therapeutic uses of PDE-III inhibitors ground to a virtual standstill. A safer PDE-III indication would thus be of tremendous benefit given how many potential disease states could be ameliorated by inhibition of PDE-III.

### SUMMARY OF THE INVENTION

Thus, and in accordance with the present invention, there is provided a method of inhibiting PDE-III in a subject comprising the oral administration to said subject of a pharmaceutical formulation that comprises enoximone wherein the enoximone is micronized into particles of less than 10 microns, and a non-ionic surfactant at approximately 66% of the formulation by weight. In contemplated embodiments, the formulation is administered in a gelcap, or it may comprise a liquid intravenous form that is injectable. It is also contemplated that enoximone could be delivered in a solid form. The formulation may comprise anywhere from 1 to 70 milligrams of enoximone, and in preferred embodiments the formulation comprises 25, 30, 35, 40, 45, 50, 55, 60, 65 or 70 milligrams of enoximone. In certain embodiments of the invention, the subject will be suffering from a disease. The diseases may comprise one or more of glaucoma or diseases of the eye wherein control of intraocular pressure would be beneficial, platelet disorders, hypercoagulation states, thrombocytosis, thrombocythemia, renal disease, renal failure, primary pulmonary hypertension (PPH), pulmonary arterial hypertension (PAH), peripheral vascular disease, stable angina, unstable angina, myocardial infarction, eclampsia, or pre-eclampsia, erectile dysfunction, asthma, bronchospastic lung disease, chronic obstructive lung disease, or gastrointestinal disorders. The non-ionic surfactant of the present invention may comprise any one of a number of different agents. Included are sorbitan esters (sorbitan monolaurate, sorbitan monooleate, sorbitan monopalmitate, sorbitan monostearate, sorbitan sesquioleate, sorbitan trioleate), ethoxylated sorbitan esters (polyethoxyethylene sorbitan fatty acid esters, polysorbates, Tween , such as Tween80), ethoxylated (polyethoxyethylene) fatty alcohols, ethoxylated (polyethoxyethylene) fatty acids, poloxamers (Pluronic™), polyglycolized glycerides (Labrasol™, Labrafil™, Gelucires™), polyoxyethylene alkyl ethers (Brij™), polyoxyethylene castor oil derivatives (Cremphor™), vitamin E TPGS (tocopheryl polyethylene glycol succinate), glyceryl monooleates, polyvinyl alcohols, and olyoxyethylene alkyl ethers. See Handbook of Pharmaceutical Excipients (2000); Handbook of Industrial Surfactants (2000); U.S. Pat. Nos. 6,254,885 and 6,596,308. The surfactant will be present in amounts exceding 40%, but may be greater than 45%, 50%, 55%, 60%, 65%, 66%, 67%, 68%, 69%, 70%, 75%, but no more than 80%.

In yet further embodiments of the invention, it is contemplated that an additional pharmaceutical composition will be given to the subject. The additional pharmaceutical composition may be selected from the group consisting of but not limited to beta blockers, anti-hypertensives, cardiotonics, anti-thrombotics, vasodilators, hormone antagonists, endothelin receptor antagonists, cytokine inhibitors/blockers, calcium channel blockers, other phosphodiesterase inhibitors, and angiotensin type 2 antagonists. In certain specific embodiments of the invention, the endothelin receptor antagonist may be either ambrisentan or darusentan.

In another embodiment of the invention the method further comprises administering the formulation to a subject at least a second time or on a daily basis. In yet further embodiments the administration may be one time, two times, three times, or four times per day.

### DESCRIPTION OF ILLUSTRATIVE EMBODIMENTS

I. The Present Invention

As discussed above, PDE-III has been implicated as a target molecule for therapy in a variety of diseases such as cardiac hypertrophy, erectile dysfunction (ED), renal diseases, gastrointestinal disorders, and a variety of vascular and circulatory disorders (Ichioka et al., 1998; Shiraishi et al., 1998; and Boldt et al., 1993).

Many of the currently used pharmacological agents have severe shortcomings in particular patient populations, and with inhibition of PDE-III being suggested as beneficial in a wide variety of disease states, an approved and safe PDE-III inhibitor would offer a new and potentially powerful tool against a variety of diseases. The availability of a new, safe and effective PDE-III inhibitor would undoubtedly benefit patients who either cannot use the pharmacological modalities presently available, or who do not receive adequate relief from those modalities.

The present invention provides just such improved PDE-III inhibitors. Specifically, the present invention provides an optimized oral enoximone formulation comprising a non-ionic surfactant (e.g., Tween-80), where the enoximone is micronized into particles uniformly less than about 10 microns, and the non-ionic surfactant comprises about 66% by weight of the formulation, for use in the treatment of various PDE-III-related disease states. The formulation may be a gel, a liquid or a solid, but in its most preferable form is a gelcap. It has been found that micron sized particles (uniformly less than 10 microns most preferably) greatly enhances bioavailability, and the presence of a surfactant may further enhance absorption and bioavailability of the drug. The micron sized particles (with or without surfactant) allows for the use of lower dose regimens, which avoids the toxicities seen in higher doses of PDE-III inhibitors.

II. Phosphodiesterases

Cyclic nucleotide second messengers (cAMP and cGMP) play a central role in signal transduction and regulation of physiologic responses. Their intracellular levels are controlled by the complex superfamily of cyclic nucleotide phosphodiesterase (PDE) enzymes. Several PDE types have been identified as therapeutic targets for a variety of diseases (Essayan, 2001). PDE's catalyze the degradation of cAMP and cGMP to the corresponding 5′ nucleotide monophosphates. Ten different PDE families have been described to date. These enzymes exist as homodimers and there is structural similarity between the different families. However, they differ in several respects like selectivity for cyclic nucleotides, sensitivity for inhibitors and activators, physiological roles and tissue distribution. Interest in these enzymes has increased of late, both within the medical community and in the general public, as a consequence of sildenafil (Viagra®), the medication recently introduced for the treatment of erectile dysfunction. Sildenafil mediates its effects by inhibiting PDE-V.

A. PDE-III

PDE-III is part of the third family of the PDE superfamily and has been found to be distributed throughout the body. Selective inhibition of PDE-III has been accomplished with various drugs (Schudt et al., 1991; Joseph, 2000), a class of which is known as positive inotropes. Positive inotropic drugs have various mechanisms of action and have been implicated for treatment in cardiovascular settings (for a review see Bristow et al., 2001).

Positive inotropes act differently from many drugs used previously, and have potential use in the treatment of any disease state where PDE-III inhibition is implicated. Intravenous inotropic agents have been used to treat cardiac emergencies and refractory heart failure, and PDE-III inhibiting drugs such as enoximone increase contractility by reducing the degradation of cAMP (Lehtonen et al., 2004). In addition, they can reduce both preload and afterload pressures via vasodilation (Borow et al., 1986). Short-term use of intravenous milrinone, another PDE-III inhibitor, has not been associated with increased mortality, showing that symptomatic benefit can be obtained when a PDE-III inhibitor is used in refractory heart failure (Baim et al., 1983). Furthermore, PDE-III inhibitors facilitate weaning from the cardiopulmonary bypass machine after cardiac surgery (Bristow et al., 2001). The pharmacokinetics of inotropic drugs might modify and prolong the response to therapy, for example, because of long-acting active metabolites (such as the sulfoxide forms of enoximone). These drugs display considerable differences in their pharmacokinetics and pharmacodynamics, and the selection of the most appropriate inotropic drug should be based on careful consideration of the clinical status of the patient and on the pharmacology of the drug.

PDE-III profiles of human cell preparations and tissues have also been analyzed by a semiquantitative method using selective PDE inhibitors and activators. Lymphocytes, alveolar macrophages and endothelial cells contain PDE III (Sly et al., 1997). PDE inhibitors have been able to inhibit PDE isoenzyme activities and functions of inflammatory cells with potency (Schudt et al., 1995). As mentioned previously, PDE-III is also known to affect platelet aggregation and PDE-III inhibitors may be of use in treating platelet disorders, coagulation and agglutination disorders (Sly et al., 1997), to alleviate the symptoms of angina (Schlepper et al., 1991), to treat renal diseases (Wang et al., 2002; Wagner et al., 1998; Tsuboi et al., 1996; and Takeda et al., 1991) or gastrointestinal disorders (Yamaura et al., 2001), or for the treatment of a variety of vascular and circulatory disorders (Ichioka et al., 1998; Shiraishi et al., 1998; and Boldt et al., 1993). PDE-III inhibition has also been shown to have potentially therapeutic benefit in the control of intraocular pressures or for the potential treatement of glaucoma and other ocular disorders (Lee et al., 1993; Mishima et al., 1991) Thus, PDE-III inhibitors such as enoximone may be beneficial in the treatment of wide variety of diseases.

B. Enoximone

Enoximone (1,3-Dihydro-4-methyl-5-[4-(methylthio)benzoyl]-2H-imidazol-2-one) is a small organic molecule that exhibits highly selective inhibition of PDE-III, both against the cAMP and cGMP conversion reactions. As heretofore mentioned, PDE-III is an enzyme that is present in the heart and plays an important regulatory role in cardiac function, thus much of the previous work on enoximone focused on cardiovascular applications. Inhibition of cardiac PDE-III increases the force of contraction of the heart, thereby increasing cardiac output. Compounds that increase the force of contraction of the heart, like enoximone, are referred to as positive inotropes. Enoximone also causes vasodilation, an increase in the diameter of blood vessels, through its effects on smooth muscle cells that surround blood vessels, which results in lower pressure against which the heart must pump. Positive inotropy and vasodilation can both be therapeutically useful in the treatment of heart failure. Enoximone is described in detail in U.S. Pat. No. 4,505,635, which is hereby incorporated by reference.

Perfan I.V.™ is an intravenous formulation of enoximone that is currently marketed in eight European countries. Clinical studies supporting the use of Perfan I.V.™ were completed in the late 1980s, and the drug was first approved in Europe in 1989. Perfan I.V.™ is used in a hospital setting to treat patients with acute decompensated heart failure (Classes III and IV) and to wean patients from cardiopulmonary bypass following open-heart surgery. This treatment, along with the use of powerful intravenous diuretics, and vasodilators, serves to increase the efficiency of the circulatory system and provide symptomatic relief to the heart failure patient. After stabilization and discharge from the hospital, patients often decompensate again within months and must be readmitted to the hospital for another round of intravenous treatment. As their disease progresses, the frequency of decompensation and hospitalization increases until patients must be maintained on continuous or intermittent treatment with these intravenous agents, which is both confining and costly.

Three Phase III trials of low-dose enoximone oral capsules are currently underway for patients with advanced chronic heart failure and one Phase III trial has been completed. In the 1980s, Merrell Dow (now part of Aventis) conducted clinical evaluation of enoximone capsules for the treatment of chronic heart failure. Enoximone capsules were evaluated in approximately 5,000 patients with chronic heart failure in multiple Phase I and Phase II clinical trials conducted in the United States, Europe and Japan. The drug was initially tested at doses now considered high—100 to 300 milligrams administered three times a day. At these high doses, patients treated with enoximone capsules demonstrated clinically significant increases in quality of life scores and maximal exercise capacity. However, in one Phase II placebo-controlled trial involving 151 patients administered enoximone capsules at doses of 100 milligrams or placebo capsules three times a day, there was a statistically significant increase in the mortality rate in the group of patients receiving enoximone capsules compared to the group receiving placebo capsules: 36% of the patients treated with enoximone capsules died during the trial versus 23% of the patients treated with placebo.

One of the inventors, Dr. Michael Bristow (Univ. of Colorado Health Science Center), continued to experiment with dosing and clinical regimens -for enoximone, and has made the unexpected observation that enoximone capsules administered at lower doses appeared to retain efficacy without increasing mortality (Bristow, 1994). Subsequently, a series of Phase II clinical trials have reported that (a) enoximone capsules administered at doses of 25 and 50 mg three times a day increased maximal exercise capacity with no apparent increase in mortality in patients with Class II and III chronic heart failure after 12 weeks of treatment (two placebo-controlled trials involving a total of 273 patients); (b) enoximone capsules administered at doses of 25 to 75 mg three times a day extended the survival times of patients with Class IV chronic heart failure awaiting a heart transplant (186-patient open-label, parallel-control trial); (c) and enoximone capsules administered at doses of 25 and 50 mg three times a day enabled patients with Class IV chronic heart failure, and otherwise too weak to tolerate beta-blockers, to receive and benefit from beta-blocker therapy. These benefits included a significant reduction in the severity of their chronic heart failure symptoms and hospitalization events (30-patient, open-label trial). In addition, Dr. Bristow has conducted a series of open-label trials of enoximone capsules involving over 200 patients to gather additional clinical data. However, the reported studies were preliminary in nature and merely constitute an experimental use of enoximone.

Another clinical trial conducted by Dr. Bristow gave rise to U.S. Pat. No. 5,998,458. This patent claims the use of positive inotropic therapy in combination with β blockade in a specific manner, including oral enoximone formulations. However, again, the described clinical trials were not sufficient to constitute anything more than an experimental use.

In June of 2000, Myogen, Inc., a company focused on cardiovascular research, initiated a Phase III program to evaluate the safety and efficacy of enoximone capsules for the long-term treatment of patients with advanced chronic heart failure. In these studies, enoximone capsules are being used in addition to standard therapies, including diuretics, ACE inhibitors and beta-blockers. The Phase III program includes four trials designed to collectively demonstrate that enoximone capsules at doses of 25 or 50 mg administered three times a day are effective in reducing hospitalizations, improving symptoms of chronic heart failure, improving quality of life and reducing the need for intravenous inotropic therapy.

EMOTE was a randomized, double-blind, placebo-controlled Phase III trial of approximately 200 patients with the most advanced stage of chronic heart failure, and who were dependent on intravenous inotrope therapy. The trial was designed to evaluate the use of enoximone capsules to wean patients off of intravenous inotrope therapy. Patients received 26 weeks of treatment. This trial was conducted in the United States and showed a statistically significant difference between drug and placebo in weaning patients off of i.v. therapy (Lalukota et al., 2004).

ESSENTIAL I is a randomized, double-blind, placebo-controlled pivotal Phase III trial of approximately 900 patients with Class III and IV chronic heart failure that are being treated with beta-blockers and other therapies according to current guidelines. The trial will track the time from randomization to cardiovascular hospitalization or death for each patient as the primary endpoint. On average, patients will receive treatment for at 1 east 12 months. This trial is being conducted in North and South America. Patient enrollment was completed in May of 2004 and results of the trial are due to be released sometime in the summer of 2005.

ESSENTIAL II is a Phase III trial identical in design and size to ESSENTIAL I. This trial is being conducted in Western and Eastern Europe. Patient enrollment was completed in May of 2004 and results of the trial are due to be released sometime in the summer of 2005.

EMPOWER is a randomized, double-blind, placebo-controlled Phase III trial of approximately 175 patients with Class III and IV chronic heart failure. Patients will be treated for 26 to 36 weeks with either (i) placebo, (ii) extended release metoprolol, a frequently prescribed beta-blocker, or (iii) extended release metoprolol in combination with enoximone capsules. The primary objective of this study is to determine whether enoximone capsules can increase the tolerability to metoprolol in patients previously shown to be intolerant to beta-blocker treatment. Patient enrollment began in September 2003.

The enoximone gelcaps used in the clinical trials, and the formulation of the current invention, comprise 25 or 50 milligrams of enoximone wherein the enoximone is present as a particle size that is uniformly less than 10 microns, and additionally the formulation may comprise 66% Tween-80 (or a similar surfactant) by weight.

i. Synthesis

Enoximone may be prepared according to the following method. A solution of 25.0 g of 4-(methylthio)-benzoic acid and 22 ml of thionyl chloride in 50 ml of benzene is refluxed for 4 hrs. Excess reagent and solvent is evaporated and the residue is azeotroped 3 times with benzene to remove all thionyl chloride. The residue is added dropwise to a mixture of 11.8 g of 1,3-dihydro-4-methyl-2H-imidazol-2-one, 40.0 g of anhydrous aluminum chloride and 100 ml of nitrobenzene. The resulting mixture is stirred at 60°-65° C. for 5 hrs, poured on ice and the precipitate that forms is collected, washed with ethyl ether and water, and recrystallized from isopropanol-water to give the title compound. M.P. 255°-258° C. (dec.).

ii. Micronized Forms

In many drug manufacturing, milling and micronizing machines pulverize substances into extremely fine particles, and thus reduce bulk chemicals to the required size for pharmaceutical formulation. The primary benefit to micronizing is the increase in solubility/bioavailability due to the increase in surface area. These finished chemicals are combined and processed further in mixing machines. The mixed ingredients may then be mechanically capsulated, pressed into tablets, or made into solutions.

Optimization and control of these processes, particularly relating to particle size, are becoming ever more important in the development of pharmaceuticals. Air jet micronization is a well proven technique that consistently produces particles in the 1-30 micron range. Micron Technologies and Jet Pharma are contract micronizers. The primary advantages of air jet micronizers are that particle reduction occurs via particle to particle collisions, with limited reduction from metal to product contact, and no generation of heat. Other advantages include no moving parts and easy to clean surfaces.

The original principles of jet milling are simple. The powder particles are fed into the flat cylindrical milling chamber tangentially through a venturi system by pressurized air or nitrogen. The particles are accelerated in a spiral movement inside the milling chamber by a number of nozzles placed around the periphery of the chamber. The micronizing effect takes place by the collision between the incoming particles and those already accelerated into the spiral path. While centrifugal force retains the larger particles at the periphery of the milling chamber, the smaller particles exit with the exhaust air from the center of the chamber. The particle size distribution is controlled by adjusting a number of parameters, two of the main ones being pressure and feed rate.

U.S. Pat. Nos. 6,645,466, 6,623,760, 6,555,135, hereby incorporated by reference, describe other micronization procedures.

III. Diseases States Treated by Inhibition of PDE-III

A. Circulatory Disorders

i. Platelet Disorders (General)

Platelets are circulating cell-derived fragments that are required for the maintenance of hemostasis. These small, anucleate fragments represent the first line of defense against hemorrhage following vascular injury, and are crucial for blood coagulation. Platelets are the terminal differentiation product of megakaryocytes, which in turn originate from pluripotent stem cells. The process of platelet production from megakaryocytes, which is complex and incompletely understood, is called thrombopoiesis. Several cytokines have been reported to stimulate the growth and maturation of megakaryocytes. The interaction between the cytokines and growth factors, their kinetic choreography, and the specific molecular steps that commit the megakaryocytes and their precursors to the process of maturation and platelet production have only begun to be rigorously investigated. Megakaryocytes mature by a process of endomitosis and cytoplasmic maturation. Most research to date has focused on the maturation step of megakaryocyte growth rather than on the terminal process of platelet production.

Morphological studies of marrow megakaryocytes suggest that platelets form as a result of cytoplasmic fragmentation. With the completion of endomitosis, megakaryocyte cytoplasm expands and, in the process, develops demarcation membranes and granules. Platelets form as the fully mature megakaryocyte develops cytoplasmic extensions, or pseudopodial protrusions, that extend in proximity to sinusoidal endothelial cells (Tavassoli and Aoki, 1989). Platelets bud from the ends of these protusions and thereafter enter the circulation. The megakaryocyte's ability to produce platelet buds is ultimately exhausted, and it undergoes terminal apoptosis.

The in vitro counterpart to thrombopoiesis is believed to be the development of the “proplatelet” process that has been observed in the terminal phases of megakaryocyte tissue cultures (Choi et al., 1995). Some data suggests that proplatelets can produce platelet-like particles (Choi et al., 1995; Zeigler et al., 1994). Proplatelets insinuating between bone marrow sinusoidal cells can enter the circulation (Tavassoli and Aoki, 1989). Circulatory shear forces within the marrow or possibly in the pulmonary circulation could result in the fragmentation of these proplatelets, thereby producing platelets in circulation (Burstein et al., 1995; Trowbridge et al., 1982).

A number of diseases or conditions result from inappropriate levels or inadequate functioning of blood platelets. Platelet disorders are clinically treated by administering thrombopoietin or by whole blood or platelet transfusions. Platelets for such procedures are obtained by plateletphoresis from normal donors; however, blood and platelet supplies can be limited. In addition, platelets have a relatively short shelf-life of about 5 days. Transfusions are also costly and can transmit infections and expose patients to viruses such as the human immunodeficiency virus (HI) or various hepatitis viruses. Furthermore, patients are often refractory to subsequent transfusions. Thrombopoietin treatment has a lag period before the level of platelets are affected and often results in the failure to stimulate platelet production in many patients. Thus, there remains a need in the art for new and improved methods of stimulating or enhancing the production of platelets in vivo, thereby resulting in safer alternatives for treating and/or preventing blood platelet disorders.

One potential method of combating platelet disorders involves inhibiting the action of PDE-III. PDE-III is present in large quantities in platelets (Sly et al., 1997) and as such is a potential therapeutic target in platelet disorders. Platelet inhibition in general has been shown to reduce the risk of ischaemic stroke, myocardial infarction, and vascular death and should be prescribed for all but those in whom it is medically contra-indicated (Samra et al., 2003). Symptom-specific pharmacotherapy with a broad range of medications has yielded disappointing results in the past. Pharmacotherapy specifically indicated for the treatment of intermittent claudication (IC), a common manifestation of peripheral arterial disease, includes selective PDE III inhibitors, which have been shown to have antiplatelet, antithrombotic, antiproliferative, and vasodilatory activity, as well as a positive effect on plasma lipids (Reilly and Mohler, 2001). Clinical studies have shown that treatment with cilostazol, a known PDE-III inhibitor, produces statistically significant increases in mean walking distance (MWD) and pain-free walking distances (PFWD) within 4 weeks, as well as improvements in functional status at 24 weeks, compared with placebo and pentoxyfylline in patients with moderate-to-severe IC (Smith, 2002). Many studies have been performed in a number of other platelet disorder disease settings which indicate or even promote the use of PDE-III inhibitors for combating these diseases (Tang et al., 1994; Pinna et al., 1997; Laguna et al., 1997; Minami et al., 1997; and Hirose et al., 2000).

ii. Hypercoagulation States

Hypercoagulation disorders (or hypercoagulable states or disorders) have the opposite effect of the more common coagulation disorders. In hypercoagulation, there is an increased tendency for clotting of the blood, which may put a patient at risk for obstruction of blood vessels (phlebitis or pulmonary embolism).

In normal hemostasis, clots form at the site of the injury. The difference between normal clotting and the clotting present in hypercoagulation is that in hypercoagulation disorders, clots can occur throughout the body's blood vessels, sometimes creating a condition known as thrombosis. Thrombosis can lead to infarction, or death of tissue, as a result of blocked blood supply to the tissue. In association with certain genetic disorders, hypercoagulation disorders may be more likely to lead to thrombosis (Penner, 1980). Hypercoagulation disorders may also be known as hyperhomocystinemia, antithrombin III deficiency, factor V leyden, and protein C or protein S deficiency.

Hypercoagulation disorders may be acquired or hereditary (Penner, 1980). Some of the genetic disorders that lead to hypercoagulation are abnormal clotting factor V, variations in fibrinogen, and deficiencies in proteins C and S. Other diseases may also cause these clotting disorders, for example diabetes, sickle cell anemia, congenital heart disease, lupus, thalassemia, polycythemia rubra vera, and others.

In order for coagulation to occur, platelets (small, round fragments in the blood) help contract blood vessels to lessen blood loss and also to help plug damaged blood vessels. However, the conversion of platelets into healthy clots is a complicated process involving a number of clotting factors. These factors are primarily found in the plasma. Proteins C and S are two of the clotting factors that help regulate or activate parts of this process. Protein C is an anticoagulant. Mutation defects in the proteins may decrease their concentrations in the blood, and may or may not affect their resulting anticoagulant activity. Factor V is an unstable clotting factor also present in plasma. Abnormal factor V resists the changes that normally occur through the influence of protein C, which can also lead to hypercoagulability. Prothrombin, a glycoprotein that converts to thrombin in the early stage of the clotting process, is affected by the presence of these proteins, as well as other clotting factors.

The diagnosis of hypercoagulation disorders is completed with a combination of physical examination, medical history, and blood tests. An accurate medical history is important to determine possible symptoms and causes of hypercoagulation disorders. There are a number of blood tests that can determine the presence or absence of proteins, clotting factors, and platelet counts in the blood. Among the tests used to detect hypercoagulation is the Antithrombin III assay. Protein C and Protein S concentrations can be diagnosed with immunoassay or plasma antigen level tests.

Coumadin and heparin anticoagulants may be administered to reduce the clotting effects and maintain fluidity in the blood. Heparin is an anticoagulant that prevents thrombus formation and is used primarily for liver and lung clots. While these treatments may be effective in certain settings, the continued problems created by these disorders has led the inventors as well as others to explore the possibility of inhibiting PDE-III for the treatment of hypercoagulation states (Hirose et al., 2000; Meanwell et al., 1992).

iii. Thrombocytosis or Thrombocythemia

Thrombocytosis is a condition marked by the absolute increase in the number of circulating platelets. In some cases the elevation is acute and transient; in others it is chronic and persistent. The term “reactive thrombocytosis” has been commonly applied to define the concept that these patients have increased circulating platelet numbers in response to some underlying disease. This is in contrast to the condition where an autonomous drive to platelet production exists, commonly termed “thrombocythemia.”

Reactive thrombocytosis may appear and persist as a result of chronic blood loss with iron deficiency, chronic inflammatory disease, chronic infectious disease, cancer and hemolytic anemia.

Primary thrombocythemia, also known as essential thrombocythemia, is an autonomous clonal proliferation of a pluripotent hematopoietic stem cell that results in an absolute increase in the number of circulating platelets. It shares several clinical features with other myeloproliferative disorders, most notably frequent bleeding and thrombotic lesions that represent major causes of morbidity and mortality.

Inhibitory factors capable of clinically significant megakaryocyte suppression have not been well-characterized. For example, both immunocytes and transforming growth factor-β (TGF-β) have been studied as potential inhibitors of megakaryocytopoiesis, with inconclusive results (Gewirtz et al., 1986). Additionally, autoregulation via negative feedback mechanisms involving megakaryocyte products, including platelet-secreted 12-17 kD glycoprotein, has been reported (Dessypris et al., 1987). Platelet factor 4 and a synthetic C-terminal peptide have been shown to be capable of inhibiting megakaryocytopoiesis (Gewirtz et al., 1989). It has also been suggested that interferon-α and interferon-γ may have a role in regulating megakaryocyte colony formation (Ganser et al., 1987; Chott et al., 1990). While interferon-α has been used to lower platelet counts in patients with primary thrombocythemia and thrombocytosis associated with other types of malignant lesions, only approximately about 50% of patients achieve a stable state of remission. Moreover, on cessation of interferon therapy, recurrence of clinical and laboratory findings is usual (Gisslinger et al., 1989).

While the potential utility of negative autocrine regulators or other megakaryocytopoiesis inhibitors in the clinical treatment of disorders characterized by excessively high platelet counts is apparent, none of the heretofore postulated inhibitors has so far proved useful in such applications. Cytoreducive chemotherapeutic agents such as alkylating agents, radiophosporous and antimetabolites have been used to reduce platelet numbers. Most have leukemogenic potential. Their use has largely been abandoned in favor of hydroxyurea. However, hydroxyurea should at best be considered an agent with uncertain carcinogenic potential because at least one case of primary thrombocythemia conversion to acute leukemia has been linked to hydroxyurea therapy (Anker-Lugtenberg et al., 1990).

Anagrelide, a member of the imidazo(2,1-b)quinazolin-2-one series, is an investigational drug which has been used for the treatment of thrombocytosis and inhibits a form of phosphdiesterase found in platelets (Pescatore and Lindley, 2000; Birgegard et al., 2004). Anagrelide has been shown to be capable of controlling platelet counts in most patients suffering from essential thrombocythemia as a consequence of an underlying myeloproliferative disorder. Suppression of platelet counts by anagralide appears to be selective relative to changes in white blood cell count and hemoglobin. However, the drug's potent effect on inhibiting platelet activation requires further study. Other investigators have explored PDE inhibiting enzymes in these disease (Meanwell et al., 1992), and it has also previously been shown that E5510, a drug used in the treatment of platelet diseases, has selective PDE-III inhibiting properties (Nagakura et al., 1996). These results all strongly implicate a possible therapeutic role for a selective PDE-III inhibitor such as enoximone in the treatment of thrombocytosis or thrombocythemia.

B. Renal Disease and Renal Failure

Renal disease, including renal failure (acute and chronic) is a common clinical problem which tends to increase with the age of humans. Conditions are described in “The Merck Manual” (16th ed., 1992), and are commonly, but not always, associated with abnormally high blood pressure (hypertension). Renal disease often results in long suffering periods where the patient endures uncomfortable and painful symptoms, often involving injury to eyes, heart and brain. Dialysis and kidney transplantation can be used as treatments if circumstances allow, but these procedures can have serious complications, including, for transplantation, organ rejection.

In animals, the underlying etiology of the disease can be uncertain, even when histopathological examination has taken place (see, e.g., Elliott and Barber, 1998; Michell, 1995). There are many commonly used measurements of renal function such as those mentioned by Finco et al. (1999)—glomerular filtration rate (GFR), plasma creatinine concentration, morphologic examination of kidney tissue, blood urea nitrogen, incidental biological events such as hypertension and proteinurea. Michell (1995) defines chronic renal failure as a “failure of clearance.” Finco (1999) suggests that declining GFR measurements are the most reliable indicator of the disease.

Treatment of renal disease associated with hypertension with antihypertensive agents has been propounded, for example with angiotensin converting enzyme (ACE) inhibitors, calcium channel blockers, etc. (see, e.g., Bright, 1999). Other treatments are mentioned by Brown (1999).

With regard to chronic renal failure associated with hypertension, treatment with amlodipine, disclosed in EP 0089167, has been previously recommended (Henik et al., 1997; Snyder et al., 1998; Cooke et al., 1998; Reams et al., 1987; and Pearce et al., 1996). Amlodipine is a dihydropyridine calcium channel blocker which is licensed for use as an antihypertensive and antianginal agent. It has also been found that calcium channel blockers such as amlodipine can be used to treat renal disease in animals which are not hypertensive, i.e., animals which are “normotensive” (U.S. Pat. No. 6,521,647). “Normotensive” means having systemic arterial blood pressure values within normal or reference ranges established for the animal species of interest, using acceptable methods for measuring such blood pressure under appropriate circumstances, and below generally accepted “hypertensive” ranges for such animals. Within an animal species, reference range values may be established for representative subclasses, races, breeds, etc. (e.g., humans, lab. animals, specific subpopulations).

Investigations of recent years revealed that isozymes of PDE are a critically important component of the cyclic-3′,5′-adenosine monophosphate (cAMP) protein kinase A (PKA) signaling pathway in the kidney (Dousa, 1999). Current evidence indicates that PDE isozymes play a role in several pathobiologic processes in kidney cells (Dousa, 1999).

In rat mesangial cells, PDE3 and PDE4 compartmentalize cAMP signaling to the PDE3-linked cAMP-PKA pathway that modulates mitogenesis and PDE4-linked cAMP-PKA pathway that modulates generation of reactive oxygen species. Administration of selective PDE isozyme inhibitors in vivo suppresses proteinuria and pathologic changes in experimental anti-Thy-1.1 mesangial proliferative glomerulonephritis in rats (Dousa, 1999). PDE isozymes also play an important role in the pathogenesis of acute renal failure of different origins. Administration of PDE isozyme-selective inhibitors suppresses some components of immune responses to allograft transplant and improves preservation and survival of transplanted organ. PDE isozymes are a target for action of numerous novel selective PDE inhibitors, which are key components in the design of novel “signal transduction” pharmacotherapies of kidney diseases (Dousa, 1999). The selective cAMP-PDE inhibitors rolipram and milrinone in combination (inhibiting PDE-IV and PDE-III isoenzymes) completely prevented hypercalcemia in an experimental model, and PDE inhibitor treatment significantly prevented the reduced expression of collecting duct aquaporins and prevented the development of polyuria associated with renal disease (Wang et al., 2002). These results all indicate a potential role for PDE-III in renal diseases and further implicate PDE-III inhibitors such as enoximone in the treatment of the diseases.

C. Cardiovascular Conditions

i. Peripheral Vascular Disease

Peripheral vascular disease, or PVD, is a condition in which the arteries that carry blood to the arms or legs become narrowed or clogged. This interferes with the normal flow of blood, sometimes causing pain but often causing no symptoms at all. The most common cause of PVD is atherosclerosis (often called hardening of the arteries). Atherosclerosis is a gradual process in which cholesterol and scar tissue build up, forming a substance called “plaque” that clogs the blood vessels. In some cases, PVD may be caused by blood clots that lodge in the arteries and restrict blood flow.

Functional peripheral vascular diseases don't have an organic cause. They don't involve defects in blood vessels' structure. They're usually short-term effects and can come and go. Raynaud's disease is an example. It can be triggered by cold temperatures, emotional stress, working with vibrating machinery or smoking.

Organic peripheral vascular diseases are caused by structural changes in the blood vessels, such as inflammation and tissue damage. Peripheral artery disease is an example. It is caused by fatty buildups in arteries.

Vascular disease of the limbs caused by organic arterial obstruction (e.g., arteriosclerosis obliterans) generally involves segmental arteriosclerotic narrowing, and the concomitant obstruction of the lumen in arteries supplying the extremities, particularly in peripheral body parts such as the limbs. In the progression of the disease, organic obstruction leads to occlusion of the artery, which in turn leads to an interruption of the vascular supply to a tissue or organ, resulting in ischemia or necrosis (Ross, 1986). PVD becomes clinically manifest usually between the ages of 50 and 70, and is more prevalent in men than in women. The lower limbs are more frequently involved than the upper limbs, and the most commonly affected vessel is the superficial femoral artery (Schadt et al., 1961).

Clinical manifestations of PVD include intermittent claudication, pain at rest, and trophic changes in the involved tissue or limb (Coffinan, 1979). A related clinical condition, Leriche's syndrome, involves isolated aortoiliac disease, and generally manifests as intermittent claudication of the lower back, buttocks, and thigh or calf muscles.

In addition, atherosclerotic PVD, involving the distal aortoiliac arteries and trauma to those vessels, are thus a common cause of vascular impotence. Individuals suffering from such vascular impotence generally have diminished or substantially absent femoral pulses, and generally present with Leriche's syndrome, although claudication may be absent in some cases. Furthermore, atherosclerotic macro- and microvascular disease are major factors contributing to erectile dysfunction in from 30 to 50 per cent of diabetic men who develop impotence.

PVD has been treated medically with some success, using agents such as pentoxifylline, which acts by increasing red cell membrane deformability, thereby reducing blood viscosity (Porter et al., 1982), although other investigators have not found such viscosity-reducing agents to be efficacious (Mashiah et al., 1978). Other approaches in the treatment of PVD have employed oral, parenteral or intravenous administration of vasodilators (Hansteen et al., 1974; Coffmann et al., 1972), L-camitine (U.S. Pat. No. 4,968,719), diuretics such as 1,3-di-n-butyl-7-(2-oxypropyl)xanthine (U.S. Pat. No. 4,784,999), xanthines and xanthine derivatives (U.S. Pat. Nos. 5,321,029 and 4,454,138), selective inhibitors of cyclic guanosine 3′,5′-monophosphate phosphodiesterase (“cGMP PDE”) (U.S. Pat. No. 5,272,147), and various classes of chromanols, chromenes and chromans having anti-hypertensive activity (U.S. Pat. No. 4,772,603). However, each approach has achieved limited success. Accordingly, there remains a need in the art to provide a more effective method of treating PVD.

PDE-III inhibitors like enoximone can satisfy that need. Pentoxifylline, for example, which is a nonspecific PDE inhibitor, has been used in the treatment of intermittent claudication and diabetes-induced peripheral vascular diseases (Angel et al., 1995). U.S. Pat. No. 6,127,541 (as well as U.S. Pat. Nos. 6,100,037; U.S. Patent 6,255,456; and U.S. Patent 6,369,059 hereinafter incorporated by reference) shows that cGMP plays an important role as a second messenger in intracellular signal transduction and an inhibitor of cGMP-specific PDE, such as enoximone, increases the concentration of intracellular cGMP, enhances the effects of endothelium-derived relaxing factor (EDRF), nitro vasodilator or atrial natriuretic peptide, shows anti-platelet activity and anti-vasocontraction activity, and also has vasodilating activity. All of these activities are useful for treating cardiovascular diseases such as peripheral vascular diseases and other related diseases like thrombosis, angina pectoris, hypertension, congestive heart failure, post-PTCA restenosis, arterial sclerosis and the like (U.S. Pat. No. 6,127,541). Thus, there is strong evidence pointing to the utility of enoximone for the treatment of PVD.

ii. Stable and Unstable Angina

Determining whether an individual is predisposed to have a stable or unstable angina condition can help individuals prepare for and prophylactically treat potentially life-threatening disease. For example, once individuals learn of their predisposition they can change their diet and daily activities such that the chance of developing an unstable angina condition is reduced. “Angina” or “angina pectoris” generally refers to chest pain resulting from an insufficient blood supply to the heart. Angina pectoris is a recurring symptom and usually occurs in the form of chest discomfort (tightness, fullness, squeezing, heaviness, burning or pain) in the center of the chest and/or over the left breast. The discomfort may move to the left shoulder and arm, although it may move to both shoulders/arms, throat, jaw, or even the lower portion of the chest or upper abdomen. It may be accompanied by shortness of breath, sweating, weakness, dizziness, nausea, or numbness in the shoulders, arms, or hands. Symptoms of angina pectoris are typically triggered by physical exertion. The symptoms are generally brief, last only 2-3 minutes and subside promptly with cessation of exercise or following the use of a nitroglycerin tablet, which typically is administered via a sublingual route. This pattern of pain is known as “stable angina.” “Chronic stable angina” generally is used to describe a patient who routinely exhibits the symptoms of “stable angina” over a prolonged period of weeks, months, or years.

While angina pectoris is rather poorly understood clinically, it is known that the resulting ischemia after cardiac insult stimulates the sensory nerves of the heart, producing the sensation of angina characterized by episodes of precordial pressure, discomfort, or a severe, intense crushing pain which may radiate to several sites including the left shoulder and left arm. Current treatments are directed to the underlying disease, usually atherosclerosis, or to drugs which either reduce myocardial oxygen demand or improve oxygen supply. Calcium antagonists such as amlodipine have been particularly useful in treating vasospastic angina, the angina of effort, and the unstable angina, due to the effect of the calcium channel antagonist on cardiac and vascular smooth muscle (U.S. Pat. No. 6,448,275).

Angina pectoris that has recently progressed or spontaneously increased in severity, frequency, or duration—particularly if accompanied by rest pain—is considered “unstable angina” (UA). Patients with the recent onset of angina, particularly if it occurs at low levels of activity or at rest, are also included in this category. Most UA patients have underlying obstructive coronary disease; the unpredictable onset of symptoms or conversion from a stable to an unstable pattern usually results from atherosclerotic plaque fissuring with superimposed platelet—or fibrin-rich thrombi. An unstable pattern can also be precipitated by extracoronary factors (secondary unstable angina). Severe anemia or carbon monoxide exposure, for example, limits blood's capacity to carry or release oxygen and can result in angina under conditions that a patient with coronary disease might otherwise tolerate well. Uncontrolled systemic arterial hypertension, rapid dysrhythmias, or hypoxemia due to pulmonary disease can also provoke angina pectoris, as can hyperthyroidism. As used herein, a patient suffering from “unstable angina” denotes a patient who has one or more of the following symptoms and signs: (1) ST segment depression, as measured by ECG; (2) slightly elevated troponin T levels, of no more than 0.1 ng/ml; or (3) slightly elevated troponin I levels, of no more than 0.4 ng/ml. In contrast to Q-wave MI, CK-MB and LDH levels are typically not elevated during UA. Also in contrast to Q-wave MI, a patient with UA typically has no ST segment elevation nor any pathological Q-wave. Finally, UA can be diagnosed solely on the basis of chest pain, typically chest pain lasting longer than 15 minutes, chest pain at rest, or chest pain following minimal exertion and that is poorly responsive to sublingual nitrates. Alternatively, even in the absence of chest pain, a patient can be diagnosed with UA if previously diagnosed with ischemic heart disease (IHD) or is considered to be at strong risk for developing IHD, and who presents with nausea, shortness of breath, palpitations, or dizziness (U.S. Pat. No. 6,706,689). Furthermore, the skilled artisan will understand that the diagnosis of UA is one of medical judgment.

Treatment of both forms of angina is accomplished with a variety of different agents. A number of investigators have examined the potential use of PDE inhibitors to treat both forms of angina (Pagani et al., 1992; U.S. Pat. Nos. 6,348,474; U.S. Patent 6,410,547). Inhibition of the cGMP form of PDE-III in particular is seen as beneficial to the alleviation of the symptoms of angina and as enoximone has such inhibitory function it is indicated for the treatment of both versions of angina.

iii. Myocardial Infarction

Ischaemic heart disease is the leading cause of death in industrialised countries. The management of ischaemic heart disease essentially relies upon one of three strategies, comprising medical therapy, percutaneous transluminal procedures, such as coronary angioplasty and atherectomy, and coronary artery bypass grafting. Although medical treatment remains the mainstay of anti-ischemic therapy, many patients undergo additional, invasive therapy in an attempt to restore coronary blood flow. However, there is increasingly intense discussion regarding not only the relative merits of these therapeutic approaches but also the point within the management of ischaemic heart disease at which they should be applied and the type of patient for which each is more appropriate.

Acute myocardial infarction (MI) strikes the majority of sufferers without prior warning and in the absence of clinically detectable predisposing risk factors (for a full review, see Braunwald, 1997). When patients come to the intensive unit in a hospital showing symptoms of acute MI, the diagnosis for acute MI requires that the patients must have (1) an increase in the plasma concentration of cardiac enzymes and (2) either a typical clinical presentation and/or typical ECG changes. Either of the following parameters will fulfill the requirement for an increase in cardiac enzymes: (1) Total creatine-kinase (CK) at least 2 times the upper limit of the normal range, or (2) CK-MB (muscle-brain) above the upper limit of the normal range and at least 5% of the normal CK. If total CK or CK-MB is not available, the following will be accepted in the fulfillment of the criteria for acute MI: (1) Troponin T at least 3 times the upper limit of the normal range; (2) Troponin I at least 3 times the upper limit of the normal range. The use of Troponin T as a serum marker for MI is disclosed in Murthy and Karmen (1997). The analytical performance and clinical utility of a sensitive immunoassay for determination of cardiac Troponin I can be taken from Davies et al. (1997).

Typical ECG changes include evolving ST-segment or T-wave changes in two or more contiguous ECG leads, the development of new pathological Q/QS waves in two or more contiguouos ECG leads, or the development of new left bundle branch block.

Secondary prevention, namely the implementation of therapy to postpone further coronary events thus continues to remain the major goal of prophylactic drug therapy in these patients.

Survivors of acute MI are at moderate risk of recurrent infarction or cardiac death. Morbidity and mortality following an MI may be related to arrhythmias, to left ventricular dysfunction, and to recurrent MI. Aspirin has been used for secondary prevention in survivors of MI. Because aspirin had a significant protective effect in secondary prevention of vascular disease, the possible benefit of aspirin in primary prevention was tested. However, several studies have shown that only a limited percent of the population at risk really benefits from aspirin therapy (Cairns et al., 1995).

The concept of secondary prevention of reinfarction and death after recovery from an MI has been actively investigated for several decades. Problems in proving the efficacy of various interventions have been related both to the ineffectiveness of certain strategies and to the difficulty in proving a benefit as mortality and morbidity have improved following MI. Although secondary prevention drug trials generally have tested one form of therapy against placebo in an attempt to demonstrate a benefit of that therapy, the physician must remember that disciplined clinical care of the individual patients is far more important than rote use of an agent found beneficial in the latest drug trial.

From an epidemiological standpoint, primary prevention is the protection of health by personal and community-wide effects such as preserving good nutritional status, physical fitness and emotional well-being. Primary prevention includes general health promotion and specific protective measures. It can also be defined as prevention of disease by altering susceptibility or reducing exposure for susceptible individuals. It is difficult to see how the administration of, for example, an angiotensin II antagonist could be viewed as a measure to promote general health. It would imply administering an angiotensin II antagonist to the population at large, with the—extremely difficult to quantify—aim of avoiding a MI in part of that population. Secondary prevention, on the other hand, includes all measures available to individuals and populations for the early detection and prompt and effective intervention to correct departures from good health. In short, secondary prevention aims to reduce prevalence by shortening the duration.ACE inhibitors have been used for secondary prevention in patients with post-MI, i.e., the use of ACE inhibitors when the patient suffers his/her first MI can prevent further complications related to the initial event and thus improve survival.

The development of the AT (1) receptor antagonists provides in addition to the ACE inhibitors a new, more specific pharmacological tool to inhibit the renin-angiotensin cascade. However, there are distinguishing features between AT (1) receptor antagonists and ACE inhibitors. One is manifested by the concomitant potentiation of bradykinin produced by ACE inhibitors, since the kinase II and converting enzyme are one in the same. The bradykinin related mechanism mediated through nitric oxide, prostaglandins, and endothelially derived hyper-polaring factor may be responsible for a different clinical effect of ACE inhibitors. Furthermore, the effect of the AT (2) is not yet clear, as an inhibition of the AT (1) receptor leads to an incease of AT (2).

Treatment, on the other hand, implies implementing measures—changes in life-style, specific drugs such as antibiotics—which can modify the course of the disease (such as administering angiotensin converting enzyme inhibitors to patients with congestive heart failure in order to prolong their survival) and/or make the cause of the disease disappear. Once the acute MI has been diagnosed, the patient can be treated with a drug which is expected to—decrease his/her mortality rate and—improve short- and long-term prognosis. The rationale behind treating patients with an acute MI rests on preliminary preclinical scientific works which have shown that the administration of compound does reduce the size of the MI, which, through its impact on left ventricular function, is one of the main determinants of survival.

Enoximone has been proven to successfully improve hemodynamics by either its positive inotropic and lusitropic properties. The expected increase in MVO₂ secondary to the increase in myocardial contractility appears to be compensated by the decrease in ventricular pre- and afterload pressure. There is a particular indication for enoximone for patients with severely impaired hemodynamics awaiting heart transplantation (“pharmacological” bridging). Promising results were documented when PDE-III-inhabitors were given in myocardial infarction and septic shock patients (Boldt et al., 1994).

iv. Eclampsia or Pre-Eclampsia

Pre-eclampsia and eclampsia are forms of high blood pressure that occur during pregnancy and are accompanied by protein in the urine and edema (swelling). As the names suggest, these two disorders are related. Pre-clampsia, sometimes called toxemia of pregnancy, may develop into the more severe eclampsia, which is pre-eclampsia together with seizure. These conditions usually develop during the second half of pregnancy (after 20 weeks), though sometimes they develop shortly after birth and, in very rare situations, they occur before 20 weeks of pregnancy. Eclampsia is the final and most severe phase of pre-eclampsia and occurs when pree-clampsia is left untreated. In addition to the previously mentioned symptoms, women with eclampsia often have seizures. Eclampsia can cause coma and even death of the mother and baby and can occur before, during or after childbirth

Pre-eclampsia affects around 5 to 10% of pregnancies. The underlying causes of pre-eclampsia remain unclear in spite of extensive clinical and basic research. Pre-eclampsia is defined in Souhami and Moxham (1994) as an abnormal rise in blood pressure between the first and second halves of pregnancy of [IE]30/20 mmHg, with abnormal urate levels of >0.35 mmol/l at 32 weeks or >0.4mmol/l thereafter, associated with proteinuria, impaired renal function and clotting disorders. The consequences of pre-eclampsia are serious and include reduced uteroplacental perfusion, fetal growth retardation, pre-term birth, and increased fetal and maternal morbidity and mortality.

The following hormones have all been identified as possible markers in an elevation of levels might be predictive of pre-eclampsia in maternal plasma: progesterone, estradiol, total human chorionic gonadotrophin (hCG), corticotrophin-releasing factor (CRF), adrenocorticotrophin (Muller et al., 1996; Ashour et al., 1997; Hsu et al., 1994; and Wenstrom et al., 1994). Conversely, levels of estriol, human placental lactogen and cortisol are unchanged or decreased. Whilst circulating CRF has been proposed as a prognostic marker for pre-eclampsia, treatment of hypertension does not influence maternal CRF levels and nor has any correlation been found between CRF levels and mean blood pressure.

In contrast with advances made in treating or eliminating many other serious disorders, severe morbidity and mortality associated with pre-eclampsia/eclampsia remain among the leading problems that threaten safe motherhood, particularly in developing countries (Villar et al., 2004). The only real known cure for preeclampsia and eclampsia is the birth of the baby. If the baby is pre-term, the condition can be managed until the baby can be safely delivered. Patients may be prescribed anti-hypertensives if the condition strikes early enough or is severe, as well as other medications to control seizures in the more severe forms of the disease. Drugs that are indicated for hypertension, such as low dose PDE-III inhibitors, could be of use in the alleviation or treatment of this disorder.

D. Erectile Dysfunction

Impotence or erectile insufficiency is a widespread disorder that is thought to affect about twelve percent of adult men under age forty-five, about twenty percent of men at age sixty, and about fifty-five percent of men at age seventy-five. Similar to male sexual dysfunction, the prevalence of female sexual dysfunction has been shown to increase with age and be associated with the presence of vascular risk factors and the development of menopause.

There is more than one cause of erectile dysfunction. For example, erectile dysfunction can be psychological, resulting from anxiety or depression, with no apparent somatic or organic impairment. Such erectile dysfunction, which is referred to as “psychogenic,” is responsible for about fifteen to twenty percent of cases of impotence. In other cases, the erectile dysfunction is associated with atherosclerosis of the arteries supplying blood to the penis; such dysfunction is referred to as “arteriogenic” or “atherosclerotic.” About forty to sixty percent of cases of impotence are arteriogenic in origin.

In still other cases, there is leakage from veins in the penis such that sufficient pressure for an erection can be neither obtained nor maintained. This dysfunction is referred to as “venous leakage,” or “abnormal drainage”. This condition is often exacerbated by the presence of some arteriogenic dysfunction whereby the supply of blood to the penis is impaired. In still other cases, the dysfunction is associated with a neuropathy, such as nerve damage arising from, for example, surgery or a pelvic injury, in the nervous system affecting the penis. Such a dysfunction is referred to as “neurogenic” and this accounts for about ten to fifteen percent of cases of impotence.

There is also a high incidence of erectile insufficiency among diabetics, particularly those with insulin-dependent diabetes mellitus. Erectile dysfunction in diabetics is often classified as “diabetogenic,” although the underlying dysfunction is usually neurogenic, but may be arteriogenic or neurogenic and arteriogenic. About half of diabetic males suffer from erectile insufficiency, and about half of the cases of neurogenic impotence are in diabetics.

Additionally, erectile insufficiency is a side effect of certain drugs, such as beta-blockers that are administered to reduce blood pressure in persons suffering from hypertension, or drugs administered to treat depression or anxiety. Excessive alcohol consumption has also been linked to erectile insufficiency. These forms of erectile insufficiency may be regarded as a subset of neurogenic or psychogenic insufficiency.

In humans, penile erection is dependent upon the relaxation of the smooth muscle tone in cells of the corpus cavemosum. This relaxation is dependent on the presence of adequate levels of a cyclic guanosine monophosphate (cyclic GMP) and cyclic adenosine monophosphate (cyclic AMP), which are regulated by phosphodiesterase (PDE) isoenzymes. Cyclic GMP and cyclic AMP are secondary messengers that can be degraded by PDE isoenzymes. The second messenger signal pathway is essential for cavernous smooth muscle relaxation.

A number of methods to treat impotence are available. These treatments include pharmacological treatments, surgery and, in cases of psychogenic dysfunction, psychological counseling is sometimes effective. In the rare cases, where the insufficiency is physical because of venous leakage, surgery can usually be employed to repair the venous lesion and thereby either cure the insufficiency or, if there remains an erectile insufficiency after repair of the venous lesion, render the insufficiency amenable to treatment by pharmacological methods.

As mentioned above, pharmacological methods of treatment are available and shown to be highly effective (U.S. Pat. No. 6,541,487). Treatments for ED include a variety of pharmacologic agents, vacuum devices, and penile prostheses. Among the pharmacologic agents, papaverine, phentolamine, and alprostadil are currently used in practice. These agents are only effective after direct intracavernosal or intraurethral injection, and are associated with side effects such as priapism, fibrosis, penile pain and hematoma at the injection site. Vacuum devices are a non-inasive alternative treatment for ED. These devices produce an erection by creating a negative pressure around the shaft of the penis resulting in an increased blood flow into the corpus cavernosum via passive arterial dilation. Although this form of therapy is frequently successful in ED of organic origin, complaints include the lack of spontaneity and the time involved in using a mechanical device, and difficulty and discomfort with ejaculation. A variety of semi-rigid or inflatable penile prostheses have been used with some success, particularly in diabetic men. These devices are generally considered when other treatment options have failed, and are associated with an increased risk of infection and ischemia.

Recently, the selective PDE-V inhibitor, sildenafil (Viagra®) was approved by the FDA as an orally effective medication for the treatment of ED. Sildenafil, 5-[2-ethoxy-5-(4-methylpiperazin-1-ylsulphonyl)phenyl]-1-methyl-3-n-propyl-6,7-dihydro-1H-pyrazolo[4,3-d]pyrimidin-7-one and a number of related analogs and their use as antianginal agents are described in U.S. Pat. Nos. 5,250,534 and 5,346,901. The use of sildenafil and related analogs for treating male erectile dysfunction is described in PCT International Application Publication No. WO 94/28902. In clinical studies, the drug improved sexual function in about 70% of the men who suffer from ED of psychogenic or organic etiology.

PDE-V is not the only PDE that is involved in erectile dysfunction. Type III PDE's, along with type V, if inhibited, are known to affect the human corpus cavernosum (Stief et al., 1998). For example, the hydrolysis of the second messenger cyclic AMP by PDE-III is known to play an important regulatory role in the relaxation of cavernous smooth muscle of the penis (Kuthe et al., 1999). Sildenafil exhibits negligible inhibition of PDE-III, the enzyme targeted by enoximone (Wallis et al., 1999). Thus, enoximone represents an attractive alternative treatment candidate over the commonly used PDE-V inhibitors on the market today for the treatment of erectile dysfunction.

E. Pulmonary Conditions

i. Pulmonary Hypertension

a. Primary (PPH)

PPH is a rare disease characterized by elevated pulmonary artery pressure with no apparent cause. PPH is also termed precapillary pulmonary hypertension or idiopathic pulmonary arterial hypertension. The diagnosis is usually made after excluding other known causes of pulmonary hypertension (Dresdale et al., 1951).

The pathophysiology of PPH is poorly understood. It is believed that an insult of some kind (e.g., hormonal, mechanical, other) to the endothelium first occurs, resulting in a cascade of events characterized by vascular scarring, endothelial dysfunction, and intimal and medial (smooth muscle) proliferation. At least 10-15% of patients with PPH have a familial form, which has only recently been characterized. Some cases may be related to sporadic genetic defects (Oudiz et al., 2004).

Early in the disease, as the pulmonary artery pressure increases and the right ventricle must perform extra work, thrombotic pulmonary arteriopathy occurs. Thrombotic pulmonary arteriopathy is characterized by in situ thrombosis of small muscular arteries of the pulmonary vasculature. In later stages, as the pulmonary pressure continues to rise, plexogenic pulmonary arteriopathy develops. This is characterized by a remodeling of the pulmonary vasculature with intimal fibrosis and replacement of normal endothelial structure (Oudiz et al., 2004).

PPH has no cure, and left untreated, PPH leads inexorably leads to right-sided heart failure and death. The overall survival rate in one study was approximately 30% at 3 years. Prior to the 1990s, therapeutic options were limited. The recent emergence of prostacyclin analogues, endothelin receptor antagonists, and other novel drug therapies has greatly improved the outlook for patients with PPH and PPH-like diseases, but no one treatment is currently considered state of the art. PDE inhibitors are being investigated for their ability to treat PPH (Kukreja et al., 2004), and PDE3 inhibitors have been shown to be potentially viable methods of treatment for PPH (Jeffrey and Wanstall, 1998; Murray et al., 2002; Tasatargil et al., 2003).

b. Secondary or PAH

Secondary pulmonary artery hypertension (SPAH) is defined as a pulmonary artery systolic pressure higher than 30 mm Hg or a pulmonary artery mean pressure higher than 20 mm Hg secondary to either a pulmonary or a cardiac disorder. If no etiology can be identified, the pulmonary arterial hypertension (PAH) is termed primary pulmonary hypertension. An increased volume of pulmonary blood flow, escalating resistance in the pulmonary vascular bed, or an elevation in pulmonary venous pressure can induce the rise in pulmonary arterial pressure (Oudiz et al., 2004).

Cardiac disorders, pulmonary disorders, or both in combination are the most common causes of secondary pulmonary hypertension. Cardiac diseases produce pulmonary hypertension via volume or pressure overload, although subsequent intimal proliferation of pulmonary resistance vessels adds an obstructive element. Perivascular parenchymal changes along with pulmonary vasoconstriction are the mechanism of pulmonary hypertension in respiratory diseases.

Therapy for secondary pulmonary hypertension is targeted at the underlying cause and its effects on the cardiovascular system. Novel therapeutic agents undergoing clinical trials have led to the possibility of specific therapies for these once untreatable disorders.

There are three predominant pathophysiologic mechanisms which may be involved in the pathogenesis of SPAH, (1) hypoxic vasoconstriction, (2) decreased area of the pulmonary vascular bed, and (3) volume/pressure overload (Oudiz et al., 2004).

Chronic hypoxemia causes pulmonary vasoconstriction by a variety of actions on pulmonary artery endothelium and smooth muscle cells, including down-regulation of endothelial nitric oxide synthetase and reduced production of the voltage-gated potassium channel alpha subunit. Chronic hypoxemia leading to pulmonary hypertension can occur in patients with chronic obstructive pulmonary disease (COPD), high-altitude disorders, and hypoventilation disorders (e.g., obstructive sleep apnea).

COPD is the most common cause of SPAH. These patients have worse 5-year survival rates, more severe ventilation perfusion mismatch, and nocturnal or exercise-induced hypoxemia. Other disorders, such as obstructive sleep apnea, neuromuscular disorders, and disorders of the chest wall, may lead to hypoxic pulmonary vasoconstriction and eventually SPAH (Oudiz et al., 2004).

A variety of causes may decrease the cross-sectional area of the pulmonary vascular bed, primarily due to disease of the lung parenchyma. The pulmonary arterial pressure rises only when the loss of the pulmonary vessels exceeds 60% of the total pulmonary vasculature. Patients with collagen vascular diseases have a high incidence of SPAH, particularly patients with systemic scleroderma or CREST (calcinosis cutis, Raynaud phenomenon, esophageal motility disorder, sclerodactyly, and telangiectasia) syndrome. A mild-to-moderate elevation in mean pulmonary artery pressure occurs secondary to acute pulmonary embolism. The peak systolic pressures usually do not rise above 50 mm Hg, and they generally normalize following appropriate therapy. Chronic pulmonary emboli can result in progressive PAH. HIV infection and several drugs and toxins are also known to cause PAH (Oudiz et al., 2004).

Disorders of the left heart may cause SPAH, resulting from volume and pressure overload. Pulmonary blood volume overload is caused by left-to-right intracardiac shunts, such as in patients with atrial or ventricular septal defects. Left atrial hypertension causes a passive rise in pulmonary arterial systolic pressure in order to maintain a driving force across the vasculature. Over time, persistent pulmonary hypertension accompanied by vasculopathy occurs. This may occur secondary to left ventricular dysfunction, mitral valvular disease, constrictive pericarditis, aortic stenosis, and cardiomyopathy (Oudiz et al, 2004).

Pulmonary venous obstruction is a rare cause of pulmonary hypertension. This may occur secondary to mediastinal fibrosis, anomalous pulmonary venous drainage, or pulmonary venoocclusive disease.

Increasing pulmonary arterial pressure is associated with a progressive decline in survival for patients with COPD or other interstitial lung diseases. The prognosis of patients with SPAH is variable and depends on the severity of hemodynamic derangement and the underlying primary disorder. Patients with severe pulmonary hypertension or right heart failure survive approximately 1 year. Patients with moderate elevations in pulmonary artery pressure (mean pressure <55 mm Hg) and preserved right heart function have a median survival of 3 years from diagnosis.

Although treatment of secondary pulmonary hypertension consists primarily of that necessary for the underlying disease, several medications and oxygen are used in different clinical settings. Currently, definite proof of effectiveness is lacking for several of these treatments (Oudiz et al., 2004). As such, there is a need for better medications for the treatment of PAH. PDE-III inhibitors have been suggested as a combination treatment in inhalants for treating pulmonary hypertension (Haraldsson et al., 2001; Schermuly et al., 2001), and could be beneficial for this disorder even as monotherapy.

ii. Asthma

Asthma is a chronic disease characterized by intermittent, reversible, widespread constriction of the airways of the lungs in response to any of a variety of stimuli which do not affect the normal lung. Estimates of the prevalence of this disease in the U.S. population range from three to six percent.

In attempting to unravel the pathogenesis of asthma, the cellular and biochemical basis (sic) for three important features of the disease have been sought: chronic airway inflammation, reversible airflow obstruction, and bronchial hyperreactivity. Theories have pointed variously to abnormalities in autonomic nervous system control of airway function, in bronchial smooth muscle contractile properties, or in the integrity of the epithelial cell lining as features that distinguish asthmatic from normal airways. Evidence suggests that the normal epithelial lining functions as more than a simple barrier: epithelial cells may produce a relaxing factor that actively maintains airway patency by causing relaxation of smooth muscle. Epithelial desquamation could contribute to bronchial hyperreactivity because a lesser amount of relaxing factor would be produced (Scientific American Medicine, 1988).

Drugs used to treat asthma fall generally into two categories: those which act mainly as inhibitors of inflammation, such as corticosteroids and cromolyn sodium, and those which act primarily as relaxants of the tracheobronchial smooth muscle, such as theophylline and its derivatives, beta-adrenergic agonists, and anticholinergics. Some of these bronchodilators may be administered orally, while others are generally given by intravenous or subcutaneous injection or by inhalation of the drug in an appropriate form, such as aerosolized powder (i.e., delivered in the form of a finely divided solid, suspended in a gas such as air), or aerosolized droplets (delivered in the form of a fine mist). Asthma patients typically self-administer bronchodilator drugs by means of a portable, metered-dose inhaler, employed as needed to quell or prevent intermittent asthma attacks (U.S. Pat. No. 5,823,180)

Current PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit all or most PDE isozyme classes in all tissues. This is a consideration in assessing the therapeutic profile of these compounds. The targeted disease state may be effectively treated by such compounds, but unwanted secondary effects may be exhibited which, if they could be avoided or minimized, would increase the overall therapeutic effect of this approach to treating certain disease states. Taken collectively, this information suggests that the side effects associated with the use of standard non-selective PDE inhibitors might be reduced by targeting novel isozyme-selective inhibitors for the predominant PDE in the tissue or cell of interest. Although in theory isozyme-selective PDE inhibitors should represent an improvement over non-selective inhibitors, the selective inhibitors tested to date are not devoid of side effects produced as an extension of inhibiting the isozyme of interest in an inappropriate or not-targeted tissue. For example, clinical studies with the selective PDE-IV inhibitor rolipram, which was being developed as an antidepressant, indicate it has psychotropic activity and produces gastrointestinal effects, e.g., pyrosis, nausea and emesis (U.S. Pat. No. 6,555,576). Type III PDE inhibitors are known to be relaxants of human airways smooth muscle (Murray et al., 1991), and as such, isozyme specific inhibitors like enoximone represent an attractive potential alternative to the treatments currently available for asthma.

iii. Bronchospastic Lung Disease

Bronchospastic disease can be a component of asthmatic diseases. They are diseases characterized by spasms of the bronchi that makes exhalation difficult and noisy; often associated with asthma and bronchitis. One approach for reversing bronchospasm and also inhibiting inflammation is to elevate intracellular adenosine cyclic 3′,5′-monophosphate (cAMP) in respiratory smooth muscle and inflammatory cells, respectively (Sutherland et al., 1968). Research has established that the xanthine-based bronchodilators, such as theophylline and aminophylline, mediate their bronchodilating activity via inhibition of cyclic AMP PDE. Agents that elevate smooth muscle cAMP concentrations induce rapid bronchodilation and inhibit the release of inflammatory mediators from activated leukocytes (Hardman, 1981; Nielson et al., 1988). By virtue of their dual mechanisms of action, such compounds can function as highly effective anti-asthmatic drugs. Enoximone is one such agent and since PDE-III inhibitors are known to relax smooth muscle of the human airways (Murray et al., 1991) they would be potentially useful agents in monotherapy or combination therapy to alleviate the symptoms of bronchospasm.

iv. Chronic Obstructive Lung Disease

Chronic obstructive pulmonary disease (COPD) is an umbrella term frequently used to describe two conditions of fixed airways disease, chronic bronchitis and emphysema, but it can also be used to describe a pulmonary syndrome that eventually leads to more advanced disease like emphysma. Chronic bronchitis and emphysema are most commonly caused by smoking; approximately 90% of patients with COPD are or were smokers. Although approximately 50% of smokers develop chronic bronchitis, only 15% of smokers develop disabling airflow obstruction. Certain animals, particularly horses, suffer from COPD as well.

The airflow obstruction associated with COPD is progressive, may be accompanied by airway hyperreactivity, and may be partially reversible. Non-specific airway hyper-responsiveness may also play a role in the development of COPD and may be predictive of an accelerated rate of decline in lung function in smokers.

COPD is a significant cause of death and disability. It is currently the fourth leading cause of death in the United States and Europe. Treatment guidelines advocate early detection and implementation of smoking cessation programs to help reduce morbidity and mortality due to the disease. However, early detection and diagnosis has been difficult for a number of reasons.

COPD takes years to develop and smokers often deny any ill effects from smoking, attributing the early warning signs of increased breathlessness as a sign of age. Similarly, acute episodes of bronchitis often are not recognized by the general practitioner as early signs of COPD. Many patients exhibit features of more than one disease (e.g., chronic bronchitis or asthmatic bronchitis) making precise diagnosis a challenge, particularly in early disease. Also, many patients do not seek medical help until they are experiencing more severe symptoms associated with reduced lung function, such as dyspnea, persistent cough, and sputum production. As a consequence, the vast majority of patients are not diagnosed or treated until they are in a more advanced stage of disease.

The use of PDE-III inhibitors has not been heavily investigated in regards to COPD. There are some reports implicating PDE-IV inhibitors in COPD (Spina, 2003; U.S. Pat. No. 6,713,509), and there is a single report indicating that PDE-III inhibition may be beneficial in treating COPD patients (Shiga et al., 2002).

F. Gastrointestinal Disorders

Finally, enoximone may be indicated for the treatment of a variety of gastrointestinal disorders. PDE-III (reported as subtype b) is abundant in bronchial, genitourinary and, most importantly, gastrointestinal smooth muscle (Reinhardt et al., 1995). It has been shown that inhibitors of PDE's can delay gastric emptying in diabetic models (Watkins et al., 2000; U.S. Pat. No. 6,451,813). Yamaura et al. (2001) have shown that treatment with PDE-III inhibitors prevents gastric intramucosal acidosis and lessens some markers of systemic inflammation. PDE-III inhibitors also slow progression of intestinal mucosal acidosis and gut barrier dysfunction (Satoh et al., 2003). Therefore, a PDE-III inhibitor like enoximone may have utility in the treatment of a variety of gastrointestinal disorders where inhibition of PDE-III is indicated.

G. Ocular Pressure Disorders

It is well recognized that regulation of aqueous humor outflow through the trabecular meshwork of the eye is critically important for maintenance of an appropriate intra-ocular pressure; and that in disease states such as ocular hypertension and glaucoma, this regulation appears to be defective. For instance, U.S. Pat. No. 4,757,089 teaches a method for increasing aqueous humor outflow by topical or intracameral administration of ethacrynic acid, or an analog thereof, to treat glaucoma. This effect is compatible with an inhibitory action at the level of mitochondrial ATP production rather than an inhibition of the Na(+)--K (+)--2Cl(−) co-transporter.

A number of hormones and neurotransmitters have been documented to decrease intra-ocular pressure by modulating aqueous production or outflow. Studies employing a human eye perfusion model have shown that epinephrine, via an apparent β-adrenergic effect upon the uvelo-scleral pathway, increases the facility of outflow. Nitrovasodilators have been found to increase outflow facility and decrease intra-ocular pressure in monkey eye. Similarly, atrial natriuretic peptide decreases intra-ocular pressure in monkey eyes and increases aqueous humor production (U.S. Patent Application 2002177625). In addition to these hormones and neurotransmitters, ethacrynic acid has been shown to increase aqueous outflow and decrease intra-ocular pressure by modulating aqueous inflow and outflow. Elevations of norepinephrine concentration in the aqueous humor resulting from cervical sympathetic nerve stimulation cause an increase in intra- ocular pressure of rabbit eye in situ by a mechanism that appears to involve an a-adrenergic effect. Similarly, topical administration of vasopressin to the eye has been shown to increase intra-ocular pressure and decrease facility of outflow in both normal and glaucomatous human eyes. A local renin-angiotensin system resides in the eye, and inhibition of angiotensin converting enzyme causes a decrease of intra-ocular pressure. In contrast to these rapidly-acting agents, administration of the glucocorticoid dexamethasone increases resistance to outflow over a slower time course of hours and days, an effect that has been postulated to occur in the expression of extracellular matrix.

Despite the large amount of work that has been done in the area of aqueous outflow regulation, more information leading to a better understanding of the regulation and to assist in the discovery of better methods of regulating intra-ocular pressure to treat diseases such as glaucoma is needed. The glaucomas comprise a heterogeneous group of eye diseases in which elevated IOP causes damage and atrophy of the optic nerve, resulting in vision loss. The underlying cause of the elevated IOP can be grossly divided into two pathophysiologic scenarios in which the drainage pathways are either physically closed off (as in the various forms of angle-closure glaucoma) or in which the drainage pathways appear anatomically normal but are physiologically dysfunctional (as in the various forms of open-angle glaucoma). Angle-closure glaucoma is nearly always a medical and/or surgical emergency, in which pharmacologic intervention is essential in controlling an acute attack, but in which the long-range management is usually surgical in nature. Primary Open Angle Glaucoma (POAG), on the other hand, has a gradual, symptomless onset and is usually treated with chronic drug therapy. POAG is the most common form of glaucoma, comprising 80% of newly-diagnosed cases in the United States and is the leading cause of blindness among African Americans.

Drugs currently used to treat glaucoma can be divided into those that reduce aqueous humor inflow and those that enhance aqueous humor outflow. The most commonly-prescribed drugs at present are the β-adrenergic antagonists, which reduce aqueous humor inflow through an unknown effect on the ciliary body. Other drugs that reduce aqueous inflow include inhibitors of carbonic anhydrase (e.g., acetazolamide and methazolamide) and the α-adrenergic agonist apraclonidine. Both of these drug classes exert their clinical effects through a poorly-understood action on the ciliary body. Each of these drugs, although effective in many patients, is poorly tolerated in some because of profound and occasionally life-threatening systemic adverse effects (U.S. Patent Application 2002177625).

Drugs that enhance aqueous humor outflow from the eye include miotics and the adrenergic agonists. The miotics exert a mechanical effect on the longitudinal muscle of the ciliary body and thus pull open the trabecular meshwork. They comprise both direct-acting parasympathomimetic agents (e.g., pilocarpine and carbachol) and indirect-acting parasympathomimetic agents (e.g., echothiopate). Miotic agents are highly effective in lowering IOP but have significant adverse effects, including chronic miosis, decreased visual acuity, painful accommodative spasm and risk of retinal detachment. Adrenergic agonists (e.g., epinephrine and dipivefrin) act on the uveoscleral outflow tract to enhance outflow through a mechanism that remains poorly understood. These drugs have perhaps the best safety profile of the compounds presently used to treat glaucoma, but are among the least effective in their IOP-lowering effect.

Accordingly, the need exists for new and better methods of lowering intra-ocular pressure, particularly in the treatment of one of the leading causes of blindness, glaucoma. As shown by Lee et al., (1993) and Mishima et al., (1991), inhibition of PDE-III can accomplish a lowering of intraocular pressure, and thus an agent like enoximone would be a viable and potentially improved therapeutic alternative for the treatment of ocular pressure disorders such as ocular hypertension or glaucoma. While it is contemplated that enoximone could be administered orally to patients suffering from ocular disorders, it is also contemplated that more standard ocular buffered formulations could be applied directly to the eye for treatment of said ocular disorders.

IV. Methods of Treatment

A. Enoximone Regimens

Currently utilized enoximone regimens comprise three daily administrations of 25 mg or 50 mg oral dosage form of enoximone. These regimens may change as needed depending on the clinical or disease state and the amount of PDE-III inhibition necessitated by the individual patient.

Initial treatment will normally begin after cessation of any prior therapies, though that may not always be the case. Combinations therapies (see below) may be commenced at such time thereafter as considered appropriate by the treating physician.

B. Combined Therapy

In certain embodiments, it is envisioned to use enoximone in combination with other therapeutic modalities. Thus, in addition to the therapies described above, one may also provide to the patient more “standard” pharmaceutical therapies. Examples of other therapies include, without limitation, so-called “beta blockers,” anti-hypertensives, cardiotonics, anti-thrombotics, vasodilators, hormone antagonists, other inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by treatment with a single composition or pharmacological formulation that includes both agents, or by treating with two distinct compositions or formulations, at the same time, wherein one composition includes enoximone and the other includes the second or additional pharmaceutical agent. Alternatively, the therapy using enoximone may precede or follow administration of the other agent(s) by intervals ranging from minutes to weeks. In embodiments where the other agent and enoximone are applied separately, one would generally ensure that a significant period of time did not expire between the time of each delivery, such that the agent and enoximone would still be able to exert an advantageously combined effect on the patient. In such instances, it is contemplated that one would typically treat with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either enoximone or the other agent will be desired. In this regard, various combinations may be employed. By way of illustration, where enoximone is “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

C. Adjunct Therapeutic Agents for Combination Therapy

Pharmacological therapeutic agents and methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

i. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

C. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

ii. Antiarteriosclerotics

Non-limiting examples of an antiarteriosclerotic include pyridinol carbamate.

iii. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

iv. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

v. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

vi. Antihypertensive Agents

Non-limiting examples of antihypertensive agents include sympatholytic, alpha/beta blockers, alpha blockers, anti-angiotensin II agents, beta blockers, calcium channel blockers, vasodilators such as phosphodiesterase inhibitors or endothelin receptor antagonists, and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-IL type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alphal-adrenergic blocking agent. Non-limiting examples of a ganglion blocking agent include mecamylamine (inversine) and trimethaphan (arfonad).

Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include ambristentan, amotriphene, bendazol, benfurodil hemisuccinate, benziodarone, bosentan, chloracizine, chromonar, clobenfurol, clonitrate, darusentan, dilazep, dipyridamole, droprenilamine, efloxate, enoximone, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, milrinone, nicorglycerin, pentaerythritol tetranitrate, pentrinitrol, perhexiline, pimefylline, sitaxsentan, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quaternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quaternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

vii. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

viii. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, anrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, peroximone, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include arninone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

ix. Additional Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents. Additionally, surgery may be used for introduction of a mechanical or cardiovascular assist device (i.e. an Acorn cardiovascular assist (CSD) device or any device mentioned below) or for installation or use of a shunt or stent.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

D. Formulations and Routes of Administration for Other Agents

While the invention is specifically directed to composition that comprises enoximone and non-ionic surfactants such as Tween-80, it will be understood that in the discussion of formulations and methods of treatment, references to compounds may also include the pharmaceutically acceptable salts, as well as alternative pharmaceutical compositions comprising metabolites or purified enantiomers of metabolites or the pharmaceutical itself (e.g., enoximone which is metabolically converted to a sulfoxide metabolite that is chiral, and thus the S or R enantiomer of that sulfoxide could be used in a pharmaceutical preparation). Where clinical applications are contemplated, pharmaceutical compositions will be prepared in a form appropriate for the intended application. Generally, this will entail preparing compositions that are essentially free of pyrogens, as well as other impurities that could be harmful to humans or animals.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles. Formulations may also be administered as nanoparticles, liposomes, granules, inhalants, nasal solutions, or intravenous admixtures

The previously mentioned formulations are all contemplated for treating patients suffering from heart failure or hypertrophy. The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Anti-Hypertensive Activity

Female spontaneously hypertensive rats were anesthetized and cannulated. The end of the cannula was exteriorized through the skin for measurement of arterial blood pressure. Approximately 30 minutes after the rats regained consciousness, the experiment began. Mean blood pressure and heart rate were recorded 15 minutes prior to drug administration, at the time of administration, and 15, 30, 45 and 60 minutes following administration. Drug was administered at a variety of dosages. A statistically significant decrease in blood pressure of ˜42% was measured at the 45 and 60 minute time points for the 100 mg/kg and 30 mg/kg groups.

### Example 2

### Anti-Hypertensive Activity

Spontaneously hypertensive rats were divided into groups and treated with vehicle or a single dose of 10, 30 or 100 mg/kg of enoximone. Mean arterial blood pressure and heart rate were recorded before and at 15, 30, 45 and 60 minutes after treatment. Blood pressure decreased in all drug treatment groups. Heart rates were unchanged in all groups, and a significant difference from vehicle was seen in lowering pressure at the 100 mg/kg dose and a non-significant but measurable lowering at the 30 mg/kg dose at 45 minutes and 60 minutes was seen (blood pressure (with vehicle=169+/−7.9; blood pressure at 45 minutes, 30 mg/kg=151+/−9; blood pressure at 60 minutes, 30 mg/kg=142+/−8; blood pressure at 45 minutes, 100 mg/kg=103+/−/.8; blood pressure at 60 minutes, 100 mg/kg=100+/−9.7).

### Example 3

### Cardiorenal Hemodynamics

Enoximone was infused i.v. at either 30 μg/kg/min or 100 μg/kg/min in anesthetized dogs. Thirty mg/kg/min showed no significant renal involvement while decreasing blood pressure and increasing cardiac contractile force. At 100 μg/kg/min, in addition to enhanced cardiotonic effects, there were measurable decreases in renal vascular resistance coupled with an increase in renal blood flow (15-20% increase in flow), while glomerular filtration was unchanged, indicating that the ability of the kidney to autoregulate was not impaired.

### Example 4

### Effects of Enoximone on Renal Function and Plasma Volume

Enoximone was administered to 7 normal human volunteers as a single I.V. dose of 2.5 mg/kg followed by repeated doses of 200 mg t.i.d. for 5 days. The results indicated no impairment of renal function while plasma volume (determined by the RISA method) increased from a baseline value of 1573+/−149.5 ml to 1900+/−79.9 ml (for a change of 327+/−135.9 ml).

### Example 5

### Enoximone and Coronary Blood Flow

Mongrel dogs were anesthetized and given bolus injections of enoximone and coronary blood flow was measured using a Stratham electromagnetic flow probe, Model SP-7516-606-214, placed around the circumflex branch of the left coronary artery near its origin. The flow probe was connected to a Stratham Model SP2202 electromagnetic flow meter which was connected to a Grass Model 7P1 low level DC preamplifier. All recordings were made on a Grass Model 7B or &d polygraph. Enoximone produced dose dependent increases in coronary blood flow (12+/−2% at 0.1 mg/kg and 41+/−6% at 1 mg/kg).

### Example 6

### Effects of Enoximone on Arteriolar Resistance—The Perfused Hindlimb

The pump perfused hindlimb preparation permits the in vivo determination of the direct vascular effects of a compound. Dogs were anesthetized and allowed to respire sponatenously. The branchial artery and vein were cannulated and a catheter was passed down the left carotid artery into the left ventricle. After dosing with enoximone (either 0.3 mg/kg or 3 mg/kg), pressure was held constant in the vascularly isolated hindlimb and blood flow was measured. Enoximone produced dose related decreases in hind limb perfusion pressure (11+/−2% and 25+/−2% respectively), an effect seen even in sympathectomized hindlimbs indicating, that these vasodilating effects occur independent of the sympathetic nervous system.

### Example 7

### Enoximone in Subjects with Angina

A double blind placebo controlled cross over trial of enoximone was done with 20 subjects displaying chronic stable angina. Enoximone as a single oral dose (75 mg) was compared with placebo. Total exercise duration was significantly longer with enoximone as compared with placebo (mean difference of 22.8 seconds), and the time to onset of angina and development of significant ST-segment decrease was similar although both showed trends in favor of enoximone.

### Example 8

### Enoximone and Human Platelet Aggregation

Substances which increase platelet cAMP levels are known to inhibit platelet aggregation. Blood was taken from human donors not on aspirin or taking any aspirin-containing substances (or other nonsteroidal anti-inflammatory drugs) for at least two weeks prior to donation. Blood was collected by venopuncture and mixed with 1 part 3.8% trisodium citrate. Platelet rich plasma (PRP) was prepared by centrifiguation at 200×g for 10 min. at room temperature. Platelet poor plasma (PPP) was prepared by centrifugation at 2000×g for 10 minutes. PRP was exposed only to plastic laboratory ware. All experiments were completed within 3 hours. PRP was incubated for 5 minutes at room temperature with enoximone prior to the addition of any aggregating agent. Aggregation was monitored by continuous recording of light transmittance in a Chrono-log dual-channel aggregometer (Chrono-log Corp.) in atotal volume of 0.5 ml PRP. Aggregating substances used were ADP (5 μM), collagen (1.0 mM) and arachidonic acid (2 μg/ml) (Chrono-log Corp.). Enoximone (0.0625-0.05 mg/ml) inhibited platelet aggregation in a concentration dependent manner. The IC50 for ADP induced aggregation was 20.9 μg/ml, 36.3 μg/ml for collagen, and was 0.240 μg/ml for arachidonic acid. It was also ruled out that enoximone was inhibiting aggregation by somehow inhibiting the metabolism of arachidonic acid as measured by HPLC and RIA.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference:
U.S. Patent App. 2002177625
U.S. Pat. No. 4,166,452
U.S. Pat. No. 4,256,108
U.S. Pat. No. 4,265,874
U.S. Pat. No. 4,454,138
U.S. Pat. No. 4,505,635
U.S. Pat. No. 4,772,603
U.S. Pat. No. 4,757,089
U.S. Pat. No. 4,784,999
U.S. Pat. No. 4,968,719
U.S. Pat. No. 5,250,534
U.S. Pat. No. 5,272,147
U.S. Pat. No. 5,321,029
U.S. Pat. No. 5,346,901
U.S. Pat. No. 5,823,180
U.S. Pat. No. 5,998,458
U.S. Pat. No. 6,100,037
U.S. Pat. No. 6,127,541
U.S. Pat. No. 6,255,456
U.S. Pat. No. 6,348,474
U.S. Pat. No. 6,369,059
U.S. Pat. No. 6,410,547
U.S. Pat. No. 6,448,275
U.S. Pat. No. 6,451,813
U.S. Pat. No. 6,521,647
U.S. Pat. No. 6,541,487
U.S. Pat. No. 6,555,135
U.S. Pat. No. 6,555,576
U.S. Pat. No. 6,623,760
U.S. Pat. No. 6,645,466
U.S. Pat. No. 6,706,689
U.S. Pat. No. 6,713,509
Anker-Lugtenberg et al., Am. J. Hematol., 33:152, 1990.
Angel et al., AIDS, 9:1137-44, 1995.
Ashour et al., Am. J. Obst. Gynecol., 176:438-444, 1997.
Asthma, Ch. 14-11 in Scientific American Medicine, 2:2,4, 1988.
Baim et al., N. Engl. J. Med., 309(13):748-56, 1983.
Birgegard et al., Haematologica, 89(5):520-527, 2004.
Boldt et al., Z. Kardiol., 83(2):75-82, 1994.
Boldt et al., Br. J. Clin. Pharmacol., 36:309-314, 1993.
Borow et al., Circulation, 73(3 Pt 2):111153-61, 1986.
Bright, Proc. 17th A CVIM, Chicago, 134-135, 1999.
Bristow et al., J. Card. Fail., 7(2 Suppl 1):8-12, 2001.
Bristow, Z. Kardiol., 83(2):15-19, 1994.
Brown, Compendium Cont. Educ. Practicing Vetn., 21:752-763, 1999.
Braunwald , Heart Disease—a Textbook of Cardiovascular Medicine, 1997.
Burstein et al., In: Magakaryopoiesis and Platelet Formation, McGraw-Hill, NY, 1995.
Cairns et al., Chest, 108(4):3805, 1995.
Choi et al., Blood, 85:402-413, 1995.
Chott et al., Br. J. Haematol., 74:10-16, 1990.
Coffman, Prog. Cardiovasc. Dis., 22:53, 1979.
Coffmann et al., Ann. Intern. Med., 76:35, 1972.
Cooke et al., J. Vet. Intern. Med., 12:123, 1998.
Davies et al., Clin. Biochem., 30:479-490, 1997.
Dessypris et al., J. Cell. Physiol., 130, 361-368, 1987.
Doherty, In: Male Infertility and Dysfunction, Hellstrom (Ed.), Chapter 34, NY, Springer-Verlag, 1997.
Dousa, Kidney Int., 55(1):29-62, 1999.
Dresdaleet al., Am. J. Med., 11(6): 686-705, 1951.
Elliott and Barber, J. Small Animal Practice, vol. 39:78, 1998. EP 0089167
Essayan, J. Allergy Clin. Immunol., 108(5):671-80, 2001.
Finco et al., J. Vet. Intern. Med., 13:516-528, 1999.
Ganser et al., Blood 70:1173-1179, 1987.
Gewirtz et al., J. Clin. Invest., 83:1477-1486, 1989.
Gewirtz et al., Blood, 68(3):619-26, 1986.
Gisslinger et al., Lancet, 1:634-637, 1989.
Goodman and Gilman's The Pharmacological Basis Of Therapeutics, Hardman et al. (Eds.), 10^{th} Ed., 32:853-860; 35:891-893, 2001.
Hansteen et al., Acta Med. Scand., 556:3-62, 1974.
Haraldsson et al., Anesth. Analg., 93(6):1439-1445, 2001.
Hardman, In: Smooth Muscle, An Assessment of Current Knowledge, Univ. of Texas Press, 1981.
Henik et al., J. Am. Animal Hosp. Assoc., 33(3):226-234, 1997.
Hirose et al., J. Cardiovasc. Pharmacol., 35(4):586-594, 2000.
Hsu et al., Am. J. Obst. Gynecol., 170:1135-1138, 1994.
Ichioka et al., Surg Res.75(1):42-48, 1998.
Jeffery and Wanstall, J. Cardiovasc. Pharmacol., 32(2):213-219, 1998.
Joseph, Toxicol Lett., 112-113:537-546, 2000.
Komas et al., In: Phosphodiesterase Inhibitors, Schudt et al., (Eds.), Ch. 6, San Diego, Calif., Academic Press, 1996.
Kukreja et al., J. Mol. Cell. Cardiol., 36(2):165-173, 2004.
Kuthe et al., Chem. Biol. Interact., 119-120:593-598, 1999.
Lehtonen et al., Clin. Pharmacokinet., 43(3):187-203, 2004.
Laguna et al., Chem. Pharm. Bull. (Tokyo), 5(7):1151-1155, 1997.
Lalukota et al., Eur. J. Heart Fail., 7:953-5, 2004.
Lee et al., Methods Find. Exp. Clin. Pharmacol., 8:527-34, 1993.
Levy et al., N. Engl. J. Med., 322:1561-1566, 1990.
Mashiah et al., Br. J. Surg., 65:342, 1978.
Meanwell et al., J. Med. Chem., 35(14):2688-2696, 1992.
The Merck Index, O'Neil et al., ed., 13^{th} Ed., 2001.
Minami et al., Life Sci., 61(25):PL 383-389, 1997.
Michell, Vet. Annual, 35:159, 1995.
Mishima et al., Curr. Eye Res., 9:817-22, 1991.
Muller et al., Am. J. Obst. Gynecol., 175:37-40, 1996.
Murray et al., Br. J. Pharmacol., 137:1187-1194, 2002.
Murray et al., Agents and Actions Supplements, 34:27-46, 1991.
Murthy and Karmen, J. Clin. Labor. Analys., 11:125-128, 1997.
Nagakura et al., Biol. Pharm. Bull., 19(6):828-833, 1996.
Nielson et al., American Rev. Respiratory Disease, 137:25, 1988.
Oudiz et al., at www.emedicine.com/med/topicl962.htm, visited May 25, 2004.
Pagani et al., Basic Res. Cardiol., 87(1):73-86, 1992.
PCT Publication WO 96/16644
PCT Publication WO 94/28902
Pearce et al., New Horizons, 4:123, 1996.
Penner, Med. Clin. North. Am., 64(4):743-59, 1980.
Pescatore and Lindley, Expert Opin. Pharmacother., 1(3):537-46, 2000.
Physician's Desk Reference, 56^{th} Ed. Medical Economics Co., Inc., NJ, 1862-1866, 2002.
Pinna et al., Farmaco., 52(1):25-28, 1997.
Porter et al., Am. Heart J., 104:66, 1982.
Reams et al., Am. J. Kidney Diseases, 6:446, 1987.
Reilly and Mohler, Ann. Pharmacother., 35: 48-56, 2001.
Reinhardt et al., J. Clin. Invest., 95(4):1528-1538, 1995.
Ross, N. Engl. J. Med., 314:488, 1986.
Samra et al., J. Ind. Med. Assoc. 101(9):561-564, 2003.
Satoh et al., Anesthesiology, 98(6):1407-14, 2003.
Schadtet al., JAMA, 175:937, 1961.
Schermuly et al., Am. J. Respir. Crit. Care. Med., 164(9):1694-700, 2001.
Schlepper et al., Z Kardiol., 80(4):75-83, 1991.
Schudt et al., Agents Actions Suppl., 34:379-40, 1991.
Shiga et al., Cardiovasc. Drugs Ther., 16(3):259-63, 2002.
Shiraishi et al., Br. J. Pharacol., 123:869-878, 1998.
Sly et al., Shock, 8:115-118, 1997.
Smith, Clin. Cardiol., 25: 91-4, 2002.
Snyder, J. Vet. Intern. Med., 12:157, 1998.
Souhami and Moxham, In: Textbook of Medicine, 2^{nd} Ed., Churchill Livingstone, 1994.
Spina, Drugs, 63(23):2575-2594, 2003.
Stief et al, J. Urol., 159(4):1390-1393, 1998.
Sutherland et al., Circulation, 37:279, 1968.
Takeda et al., Endocrinology, 129:287-294, 1991.
Tang et al., Eur. J. Pharmacol., 15;268(1):105-14, 1994.
Tasatargil et al., Auton. Autacoid. Pharmacol., 23(2):117-124, 2003.
Tavassoli and Aoki, Blood Cells, 15:3-14, 1989.
The Merck Index, O'Neil et al., ed., 13^{th} ed., 2001.
The Merck Manual, 16th edition, 149:1661-1665, 1992.
Trowbridge et al., Thromb. Res., 28:461-475, 1982.
Tsuboi et al., J. Clin. Invest., 98:262-270, 1996.
Villar et al., Int. J. Gynaecol. Obstet., 85(1):S28-41, 2004.
Wagner et al., Kidney Int. Suppl., 67:S78-83, 1998.
Wallis et al., Am. J. Cardiol., 83(5A):3C-12C, 1999.
Wang et al., Am. J. Physiol. Renal. Physiol., 283:F1313-1325, 2002.
Watkins et al., J. Clin. Invest., 106:373-384, 2000.
Wenstrom et al., A. J. Obst. Gynecol., 171:1038-1041, 1994.
Yamaura et al., Acta Anaesthesiol Scand. 45(4):427-434, 2001.
Zeigler et al., Blood, 84:4045-4052, 1994.

What is claimed:

1. A method of inhibiting PDE-III in a subject comprising administering to said subject a pharmaceutical formulation comprising enoximone; wherein enoximone is micronized into uniform particles of less than about 10 microns and a surfactant comprises about 66% by weight of the formulation.

2. The method of claim 1, wherein the formulation is administered orally as a gelcap.

3. The method of claim 1, wherein the pharmaceutical formulation comprises enoximone in a liquid form suitable for i.v. injection or for use topically in the eye.

4. The method of claim 1, wherein the formulation comprises about 10-70 milligrams of enoximone.

5. The method of claim 4, wherein the formulation comprises about 25 mg of enoximone, 30 mg of enoximone, 35 mg of enoximone, 40 mg of enoximone, 45 mg of enoximone, 50 mg of enoximone, 55 mg of enoximone, 60 mg of enoximone, 65 mg of enoximone, or 70 mg of enoximone.

6. The method of claim 1, wherein said subject is diseased.

7. The method of claim 6, wherein said disease is selected from one or more of abnormal ocular pressure disorders, glaucoma, platelet disorder, hypercoagulation states, thrombocytosis, thrombocythemia, renal disease, renal failure, PPH, PAH, peripheral vascular disease, stable angina, unstable angina, myocardial infarction, eclampsia, or pre-eclampsia, erectile dysfunction, asthma, bronchospastic lung disease, chronic obstructive lung disease, or gastrointestinal disorders.

8. The method of claim 1, further comprising providing an additional pharmaceutical composition to said subject.

9. The method of claim 8, wherein said additional pharmaceutical composition is selected from one or more of the group consisting of beta blockers, anti-hypertensives, cardiotonics, anti-thrombotics, vasodilators, hormone antagonists, endothelin receptor antagonists, vasodilators, prostenoids, prostacyclins, cytokine inhibitors/blockers, calcium channel blockers, other phosphodiesterase inhibitors, and angiotensin type 2 antagonists.

10. The method of claim 9, wherein said endothelin receptor antagonist is ambrisentan, darusentan, sitaxsentan, or bosentan.

11. The method of claim 8, wherein said additional pharmaceutical composition comprises an endothelin receptor antagonist and a (a) vasodilator or (b) venodilator.

12. The method of claim 1, further comprising administering the formulation to said subject more than one time.

13. The method of claim 12, wherein said subject receives the formulation on a daily basis.

14. The method of claim 13, wherein said subject receives the formulation 1 time, 2 times, 3 times, or 4 times a day.

15. The method of claim 9, wherein said additional pharmaceutical composition comprises esmolol, iloprost, or beraprost.

16. The method of claim 1, further comprising the use of a cardiovascular assist device.

17. A method of controlling intraocular eye pressure in a subject comprising administering to said subject an ocular pharmaceutical formulation comprising enoximone.

18. A method of treating glaucoma or ocular hypertension in a subject comprising administering to said subject an ocular pharmaceutical formulation comprising enoximone.

19. A method of inhibiting PDE-III in a subject comprising administering to said subject a pharmaceutical formulation comprising enoximone; wherein enoximone is micronized into uniform particles of less than about 10 microns.

20. The method of claim 19, wherein the formulation is administered orally as a gelcap.

21. The method of claim 19, wherein the pharmaceutical formulation comprises enoximone in a liquid form suitable for i.v. injection or for use topically in the eye.

22. The method of claim 19, wherein the formulation comprises about 10-70 milligrams of enoximone.

23. The method of claim 22, wherein the formulation comprises about 25 mg of enoximone, 30 mg of enoximone, 35 mg of enoximone, 40 mg of enoximone, 45 mg of enoximone, 50 mg of enoximone, 55 mg of enoximone, 60 mg of enoximone, 65 mg of enoximone, or 70 mg of enoximone.

24. The method of claim 19, wherein said subject is diseased.

25. The method of claim 24, wherein said disease is selected from one or more of abnormal ocular pressure disorders, glaucoma, platelet disorder, hypercoagulation states, thrombocytosis, thrombocythemia, renal disease, renal failure, PPH, PAH, peripheral vascular disease, stable angina, unstable angina, myocardial infarction, eclampsia, or pre-eclampsia, erectile dysfunction, asthma, bronchospastic lung disease, chronic obstructive lung disease, or gastrointestinal disorders.

26. The method of claim 19, further comprising providing an additional pharmaceutical composition to said subject.

27. The method of claim 26, wherein said additional pharmaceutical composition is selected from one or more of the group consisting of beta blockers, anti-hypertensives, cardiotonics, anti-thrombotics, vasodilators, hormone antagonists, endothelin receptor antagonists, vasodilators, prostenoids, prostacyclins, cytokine inhibitors/blockers, calcium channel blockers, other phosphodiesterase inhibitors, and angiotensin type 2 antagonists.

28. The method of claim 27, wherein said endothelin receptor antagonist is ambrisentan, darusentan, sitaxsentan, or bosentan.

29. The method of claim 26, wherein said additional pharmaceutical composition comprises an endothelin receptor antagonist and a (a) vasodilator or (b) venodilator.

30. The method of claim 19, further comprising administering the formulation to said subject more than one time.

31. The method of claim 30, wherein said subject receives the formulation on a daily basis.

32. The method of claim 30, wherein said subject receives the formulation 1 time, 2 times, 3 times, or 4 times a day.

33. The method of claim 27, wherein said additional pharmaceutical composition comprises esmolol, iloprost, or beraprost.

34. The method of claim 19, further comprising the use of a cardiovascular assist device.

35. The method of claim 1, wherein said surfactant is Tween-80.
